(19) United States
(12) Patent Application Publication
(10) Pub. No.: US 2023/0220036 A1
(43) Pub. Date: Jul. 13, 2023

(54) IMPROVED PROCESS FOR THE PREPARATION OF SEMAGLUTIDE

(71) Applicant: NEULAND LABORATORIES LIMITED, Hyderabad (IN)

(72) Inventors: Khalid Anwer MOHAMMED, Woodland, CA (US); Rehana BEGUM, Secunderabad (IN); Shavali SHAIK, Atmakur (IN); Kalesha SHAIK, Andhra Pradesh (IN); Sunil Kumar GANDAVADI, Hyderabad (IN); Mohammed Sharif SHAIK, Prakasam (IN); Mohosin LAYEK, Burdwan (IN); Mahender Rao SIRIPRAGADA, Hyderabad (IN); Pavan Kumar JUJJURI, Mahabubabad (IN); Hema Sundara Rao TELAKALA, Andhra Pradesh (IN)

(21) Appl. No.: 17/997,852

(22) PCT Filed: Apr. 30, 2021
(86) PCT No.: PCT/IN2021/050425

§ 371 Date: Nov. 3, 2022

(30) Foreign Application Priority Data
May 5, 2020 (IN) 202041019091
Apr. 2, 2021 (IN) 202141015767

(51) Int. Cl. C07K 14/605

(52) U.S. Cl. CPC C07K 14/605

(57) ABSTRACT

Improved process for the preparation of Semaglutide having the structural formula (I).

His-Aib-Glu-Gly-Thr-Phe-Thr-Ser-Asp-Val-Ser-Ser-Tyr-Leu-Glu-Gly-Gln-Ala-Ala-Lys (C18 di acid mono-t-butyl-γ-Glu(AEEA-AEEA)-OtBu)-Glu-Phe-Ile-Ala-Trp-Leu-Val-Arg-Gly-Arg-Gly-OH Formula-I

The present invention relates to the following fragments which are useful in the preparation of Semaglutide.

Fragment-1: Boc-His(X)-Aib-Glu(OtBu)-Gly-OH; wherein X is Boc or Trt
Fragment-2: Fmoc-Thr(tBu)-Phe-Thr(tBu)-Ser(tBu)-Asp(OtBu)-Val-Ser(Oxa)-OH
Fragment-3: Fmoc-Ser(tBu)-Tyr(tBu)-Leu-Glu(OtBu)-Gly-OH
Fragment-4: Fmoc-Gln(Trt)-Ala-Ala-Lys(PEG-PEG-γ-Glu-octadecane dioic acid)-Glu(OtBu)-Phe-Ile-Ala-Trp(Boc)-OH
Fragment-5: Leu-Val-Arg(pbf)-Gly-Arg(pbf)-Gly-OtBu
Fragment-6: H-Gln(Trt)-Ala-Ala-Lys(C18diacid mono-t-butyl-γ-Glu(AEEA-AEEA)-OtBu)-Glu (OtBu)-Phe-Ile-Ala-Trp(Boc)-Leu-Val-Arg(Pbf)-Gly-Arg(Pbf)-Gly-OtBu
Fragment-7: Boc-His(X)-Aib-Glu(OtBu)-Gly-Thr(tBu)-Phe-Thr(tBu)-Ser(tBu)-Asp (OtBu)-Val-Ser(Oxa)-OH; wherein X is Boc or Trt

The present invention also relates to novel fragment-4 which is useful in the preparation of Semaglutide.

Fmoc-Gln(Trt)-Ala-Ala-Lys(C18 diacid mono-t-butyl-γ-Glu(AEEA-AEEA)-OtBu)-Glu (OtBu)-Phe-Ile-Ala-Trp(Boc)-OH (fragment-4)

### CROSS-REFERENCE TO RELATED APPLICATIONS

This application is a National Stage application of International Patent Application No. PCT/IN2021/050425, filed on Apr. 30, 2021, which claims priority to Indian Patent Applications No. 202141015767, filed on Apr. 2, 2021, and No. 202041019091, filed on May 5, 2020, each of which is hereby incorporated by reference in its entirety.

### TECHNICAL FIELD

The present invention relates to an improved process for the preparation of Semaglutide having the sequence chemical formula (I).

His-Aib-Glu-Gly-Thr-Phe-Thr-Ser-Asp-Val-Ser-Ser-Tyr-Leu-Glu-Gly-Gln-Ala-Ala-Lys (C18 diacid mono-t-butyl-γ-Glu(AEEA-AEEA)-OtBu)-Glu-Phe-Ile-Ala-Trp-Leu-Val-Arg-Gly-Arg-Gly-OH Formula-I

The present invention also relates to novel fragment-4 which is useful in the preparation of Semaglutide.

Fmoc-Gln(Trt)-Ala-Ala-Lys(C18 diacid mono-t-butyl-γ-Glu(AEEA-AEEA)-OtBu)-Glu (OtBu)-Phe-Ile-Ala-Trp(Boc)-OH (Fragment-4)

### BACKGROUND

Semaglutide is a long-acting glucagon like peptide agonist developed by Novo Nordisk and approved by Food and Drug Administration for the treatment of type-2 diabetes. Semaglutide marketed under brand name “Ozempic” in the form of injection and “RYBELSUS” in the form of tablet which lowers the blood sugar level by increasing the production of insulin.

The Semaglutide peptide is chemically similar to Liraglutide, with the inclusion of two structural modifications. The first is replacement of Ala with the non-proteinogenic amino acid 2-aminoisobutyric acid (Aib) at position 2. The second is the attachment of octadecanoic diacid to the side chain of Lys-26 through a short polyethylene glycol (PEG) spacer and a γ-glutamic acid linker.

Semaglutide and its process for the preparation is first disclosed in U.S. Pat. No. 8,129,343. In this process, there is a possibility of formation of several impurities which shows impact on yield as well as purity of final API and additional purification techniques required to get pure Semaglutide. This process is highly expensive and commercially not viable.

WO2017114191, CN 103848910, CN 104356224, CN 108203462 and CN 108059666 also discloses the process for the preparation of Semaglutide. These processes have several disadvantages with lot of technical difficulties, expensive production costs and not suitable for large scale production due to complex purification methods.

In view of all these disadvantages, there is a significant need to develop a cost effective, stable, commercially viable, large scale and robust process for the preparation of highly pure Semaglutide with good yield.

### SUMMARY

The present invention provides an improved process for the preparation of Semaglutide by a hybrid approach.

The present invention provides a cost effective, novel and an efficient process for the preparation of Semaglutide by making appropriate fragments in a solid phase approach followed by condensing these fragments using solution phase approach with higher yields and purity.

In one embodiment, the present invention relates to an improved process for the preparation of Semaglutide by using three or four or five fragments through hybrid approach. This process will involve the coupling of appropriate fragments which are synthesised on solid support in a required sequence, deprotection and condensing them in solution phase, followed by purification on reverse phase HPLC, freeze drying and isolation to get pure Semaglutide.

The present invention provides a hybrid approach for the preparation of Semaglutide compound of formula-I.

His-Aib-Glu-Gly-Thr-Phe-Thr-Ser-Asp-Val-Ser-Ser-Tyr-Leu-Glu-Gly-Gln-Ala-Ala-Lys (C18 diacid mono-t-butyl-γ-Glu(AEEA-AEEA)-OtBu)-Glu-Phe-Ile-Ala-Trp-Leu-Val-Arg-Gly-Arg-Gly-OH Formula-I

which comprises:
a) synthesis of fragments-3, -6 and -7 on solid support;
   Boc-His(X)-Aib-Glu(OtBu)-Gly-Thr(tBu)-Phe-Thr(tBu)-Ser(tBu)-Asp(OtBu)-Val-Ser(Oxa)-OH (Fragment-7); wherein X is Boc or Trt
   Fmoc-Ser(tBu)-Tyr(tBu)-Leu-Glu(OtBu)-Gly-OH (Fragment-3)
   H-Gln(Trt)-Ala-Ala-Lys(C18diacidmono-t-butyl-γ-Glu(AEEA-AEEA)-OtBu)-Glu (OtBu)-Phe-Ile-Ala-Trp(Boc)-Leu-Val-Arg(Pbf)-Gly-Arg(Pbf)-Gly-OtBu (Fragment-6)
b) condensing H-Gln(Trt)-Ala-Ala-Lys(C18diacidmono-t-butyl-γ-Glu(AEEA-AEEA)-OtBu)-Glu(OtBu)-Phe-Ile-Ala-Trp(Boc)-Leu-Val-Arg(Pbf)-Gly-Arg(Pbf)-Gly-OtBu (Fragment-6) with Fmoc-Ser(tBu)-Tyr(tBu)-Leu-Glu(OtBu)-Gly-OH (Fragment-3) in presence of coupling agent and solvent in in-situ manner, followed by deprotection in presence of base to obtain H-Ser(tBu)-Tyr(tBu)-Leu-Glu(OtBu)-Gly-Gln(Trt)-Ala-Ala-Lys(C18diacidmono-t-butyl-γ-Glu(AEEA-AEEA)-OtBu)-Glu(OtBu)-Phe-Ile-Ala-Trp (Boc)-Leu-Val-Arg(pbf)-Gly-Arg (pbf)-Gly-OtBu;
c) condensing Boc-His(X)-Aib-Glu(OtBu)-Gly-Thr(tBu)-Phe-Thr(tBu)-Ser(tBu)-Asp (OtBu)-Val-Ser(Oxa)-OH (Fragment-7) with peptide obtained in step-b) in presence of a coupling agent to obtain protected Semaglutide;
d) cleaving the protected Semaglutide using a reagent to obtain crude Semaglutide;
e) purifying the crude Semaglutide by preparative HPLC to obtain pure Semaglutide.

The present invention provides a hybrid approach for the preparation of Semaglutide compound of formula-I.

His-Aib-Glu-Gly-Thr-Phe-Thr-Ser-Asp-Val-Ser-Ser-Tyr-Leu-Glu-Gly-Gln-Ala-Ala-Lys (C18 diacid mono-t-butyl-γ-Glu(AEEA-AEEA)-OtBu)-Glu-Phe-Ile-Ala-Trp-Leu-Val-Arg-Gly-Arg-Gly-OH Formula-I

which comprises:
a) synthesis of fragments-1, -2, -3 and -6 on solid support;
   Boc-His(X)-Aib-Glu(OtBu)-Gly-OH (fragment-1); wherein X is Boc or Trt
   Fmoc-Thr(tBu)-Phe-Thr(tBu)-Ser(tBu)-Asp(OtBu)-Val-Ser(Oxa)-OH (fragment-2)
   Fmoc-Ser(tBu)-Tyr(tBu)-Leu-Glu(OtBu)-Gly-OH (fragment-3)
   H-Gln(Trt)-Ala-Ala-Lys(C18diacidmono-t-butyl-γ-Glu(AEEA-AEEA)-OtBu)-Glu (OtBu)-Phe-Ile-Ala-Trp(Boc)-Leu-Val-Arg (Pbf)-Gly-Arg(Pbf)-Gly-OtBu (fragment-6)
b) condensing H-Gln(Trt)-Ala-Ala-Lys(C18diacidmono-t-butyl-γ-Glu(AEEA-AEEA)-OtBu)-Glu(OtBu)-Phe-Ile-Ala-Trp(Boc)-Leu-Val-Arg(Pbf)-Gly-Arg(Pbf)-Gly-OtBu (fragment-6) with Fmoc-Ser(tBu)-Tyr(tBu)-Leu-Glu(OtBu)-Gly-OH (fragment-3) in presence of coupling agent and solvent in in-situ manner, followed by deprotection in presence of base to obtain H-Ser(tBu)-Tyr(tBu)-Leu-Glu(OtBu)-Gly-Gln(Trt)-Ala-Ala-Lys(C18diacidmono-t-butyl-γ-Glu(AEEA-AEEA)-OtBu)-Glu(OtBu)-Phe-Ile-Ala-Trp (Boc)-Leu-Val-Arg(pbf)-Gly-Arg (pbf)-Gly-OtBu;
c) condensing Fmoc-Thr(tBu)-Phe-Thr(tBu)-Ser(tBu)-Asp(OtBu)-Val-Ser(Oxa)-OH (fragment-2) with peptide obtained in step-b) in presence of a coupling agent in in-situ manner followed by deprotection in presence of base to obtain H-Thr(tBu)-Phe-Thr(tBu)-Ser(tBu)-Asp(OtBu)-Val-Ser(Oxa)-Ser(tBu)-Tyr(tBu)-Leu-Glu(OtBu)-Gly-Gln(Trt)-Ala-Ala-Lys(C18diacidmono-t-butyl-γ-Glu(AEEA-AEEA)-OtBu)-Glu(OtBu)-Phe-Ile-Ala-Trp(Boc)-Leu-Val-Arg(pbf)-Gly-Arg (pbf)-Gly-OtBu;
d) condensing Boc-His(X)-Aib-Glu(OtBu)-Gly-OH (fragment-1) with peptide obtained in step-c) in presence of a coupling agent in in-situ manner followed by deprotection in presence of base to obtain protected Semaglutide;
e) cleaving the protected Semaglutide using a reagent to obtain crude Semaglutide;
f) purifying the crude Semaglutide by preparative HPLC to obtain pure Semaglutide.

The present invention provides a hybrid approach for the preparation of Semaglutide compound of formula-I.

His-Aib-Glu-Gly-Thr-Phe-Thr-Ser-Asp-Val-Ser-Ser-Tyr-Leu-Glu-Gly-Gln-Ala-Ala-Lys(PEG-PEG-γ-Glu-octadecane dioic acid)-Glu-Phe-Ile-Ala-Trp-Leu-Val-Arg-Gly-Arg-Gly-OH Formula-I

which comprises:
a) synthesis of fragments-1, -2, -3, -4 and -5 on solid support;
   Boc-His(Trt)-Aib-Glu(OtBu)-Gly-OH (fragment-1)
   Fmoc-Thr(tBu)-Phe-Thr(tBu)-Ser(tBu)-Asp(OtBu)-Val-Ser(Oxa)-OH (fragment-2)
   Fmoc-Ser(tBu)-Tyr(tBu)-Leu-Glu(OtBu)-Gly-OH (fragment-3)
   Fmoc-Gln(Trt)-Ala-Ala-Lys(C18 diacid mono-t-butyl-γ-Glu(AEEA-AEEA)-OtBu)-Glu (OtBu)-Phe-Ile-Ala-Trp(Boc)-OH (fragment-4)
   Fmoc-Leu-Val-Arg(pbf)-Gly-Arg(pbf)-Gly-OtBu (fragment-5)
b) condensing Fmoc-Leu-Val-Arg(pbf)-Gly-Arg(pbf)-Gly-OtBu (fragment-5) with Fmoc-Gln(Trt)-Ala-Ala-Lys(C18 diacid mono-t-butyl-γ-Glu(AEEA-AEEA)-OtBu)-Glu (OtBu)-Phe-Ile-Ala-Trp(Boc)-OH (fragment-4) in presence of coupling agent and solvent in in-situ manner followed by deprotection in presence of base to obtain 15 amino acid peptide chain.
c) condensing Fmoc-Ser(tBu)-Tyr(tBu)-Leu-Glu(OtBu)-Gly-OH (fragment-3) with 15 amino acid peptide chain obtained in step-b) in presence of a coupling agent followed by deprotection in presence of a base to obtain 20 amino acid peptide chain;
d) condensing Fmoc-Thr(tBu)-Phe-Thr(tBu)-Ser(tBu)-Asp(OtBu)-Val-Ser(Oxa)-OH (fragment-2) with 20 amino acid peptide chain obtained in step-c) in presence of a coupling agent followed by deprotection in presence of a base to obtain 27 amino acid peptide chain;
e) condensing Boc-His(Trt)-Aib-Glu(OtBu)-Gly-OH (fragment-1) with 27 amino acid peptide chain obtained in stage-d) in presence of a coupling agent to obtain protected Semaglutide;
f) cleaving the protected Semaglutide using a reagent to obtain crude Semaglutide;
g) purifying the crude Semaglutide by preparative HPLC to obtain pure Semaglutide.

The present invention relates to novel fragment-4 which is useful in the preparation of Semaglutide.

Fmoc-Gln(Trt)-Ala-Ala-Lys(C18 diacid mono-t-butyl-γ-Glu(AEEA-AEEA)-OtBu)-Glu (OtBu)-Phe-Ile-Ala-Trp(Boc)-OH (fragment-4)

The present invention provides a solid phase peptide process for the preparation of Fmoc-Gln(Trt) Ala-Ala-Lys(C18 diacid mono-t-butyl-γ-Glu(AEEA-AEEA)-OtBu)-Glu(OtBu)-Phe-Ile-Ala-Trp (Boc)-OH of fragment-4

which comprises:
a) anchoring Fmoc-Trp(Boc)-OH to a resin in presence of a coupling agent;
b) selective deprotection of amino acid using a base;
c) coupling of Fmoc-Ala-OH to a resin obtained in step-b) in presence of coupling agent in a solvent to obtain dipeptide resin;
d) sequential coupling of Fmoc-Ile-OH, Fmoc-Phe-OH, Fmoc-Glu(OtBu)-OH, Fmoc-Lys(PEG-PEG-γ-Glu-octadecane dioic acid)-OH, Fmoc-Ala-OH, Fmoc-Ala-OH, Fmoc-Gln(Trt)-OH to the obtained resin in step-a) in presence of a coupling agent;
e) cleaving of protected peptide from solid support resin in presence of a reagent to get fragment-4.

### Abbreviations

Fmoc: 9-fluorenylmethoxycarbonyl

### Boc: Tert-butoxycarbonyl

DCM: dichloromethane
DMF: N, N-dimethyl formamide
DIC: N,N′-diisopropyl carbodiimide

### DIEA: Diisopropylethylamine

HOBt: N-hydroxy benzotriazole
CTC resin: 2-Chlorotrityl chloride resin
SPPS: Solid phase peptide synthesis
TFA: Trifluoroacetic acid
TIPS: Triisopropyl silane
EDC·HCl: 1-(dimethylaminopropyl)-3-ethylcarbodiimide hydrochloride

### DTT: Dithiothreitol

### EDT: 1,2-Ethanedithiol

### DETAILED DESCRIPTION

The present invention provides an improved process for the preparation of Semaglutide by making appropriate fragments on solid support, followed by condensing these fragments using solution phase approach with higher yields and purity.

Peptide fragments which are used in the preparation of Semaglutide are as follows.

Fragment-1: Boc-His(X)-Aib-Glu(OtBu)-Gly-OH; wherein X is Boc or Trt

### Fragment-2: Fmoc-Thr(tBu)-Phe-Thr(tBu)-Ser(tBu)-Asp(OtBu)-Val-Ser(Oxa)-OH

### Fragment-3: Fmoc-Ser(tBu)-Tyr(tBu)-Leu-Glu(OtBu)-Gly-OH

Fragment-4: Fmoc-Gln(Trt)-Ala-Ala-Lys(PEG-PEG-γ-Glu-octadecane dioic acid)-Glu(OtBu)-Phe-Ile-Ala-Trp(Boc)-OH

### Fragment-5: Leu-Val-Arg(pbf)-Gly-Arg(pbf)-Gly-OtBu

Fragment-6: H-Gln(Trt)-Ala-Ala-Lys(C18diacidmono-t-butyl-γ-Glu(AEEA-AEEA)-OtBu)-Glu(OtBu)-Phe-Ile-Ala-Trp (Boc)-Leu-Val-Arg(Pbf)-Gly-Arg(Pbf)-Gly-OtBu
Fragment-7: Boc-His(X)-Aib-Glu(OtBu)-Gly-Thr(tBu)-Phe-Thr(tBu)-Ser(tBu)-Asp(OtBu)-Val-Ser(Oxa)-OH; wherein X is Boc or Trt

Peptide fragments are prepared by using solid phase peptide synthesis through linear approach.

Solid phase peptide synthesis is carried out on an insoluble polymer which is acid sensitive. Acid sensitive resin is selected from the group consisting of chloro trityl resin (CTC), wang resin, 4-methyltrityl chloride and rink acid resin. Preferably using CTC resin. The resin used for the synthesis of Semaglutide undergoes swelling in presence of a solvent selected from the group consisting of dichloromethane, N, N-Dimethylformamide and N-methyl-2-pyrrolidone or its mixture.

Coupling of amino acid to a resin is carried out in presence of a base. The base is organic or inorganic base. The inorganic base is selected from the group consisting of potassium carbonate, lithium carbonate, sodium carbonate, sodium bicarbonate, potassium bicarbonate, sodium hydroxide, potassium hydroxide, ammonium hydroxide and mixture thereof; the organic base is selected from the group consisting of diisopropyl amine, N, N-diisopropyl ethylamine, triethylamine, tertiary butyl amine, dimethylamine, tri methyl amine, isopropyl ethylamine, pyridine, N-methyl morpholine and mixture thereof.

Solvents used in this coupling reaction is selected from the group consisting of DMF, DCM, tetrahydrofuran, NMP, DMAC, methanol, ethanol, isopropanol, dichloroethane, 1,4-dioxane, ethyl acetate, acetonitrile, acetone or a mixture thereof.

According to the present invention, the cleavage and global deprotection of the peptide is carried out with a cocktail mixture. The cleavage of peptide from resin involves treating the protected peptide anchored to a resin with an acid having at least a scavenger. The acid used in the cleavage is trifluoro acetic acid. The scavengers used are selected from the group consisting of TIPS, phenol, thioanisole, water or mixture thereof. Preferably using a cocktail mixture of TFA, TIPS, water and DTT (90%: 5%: 5%: 2.5%).

In the present invention, isolation of Semaglutide is carried out by precipitating with ether solvent. Ether solvent used in this reaction is selected from the group consisting of methyl tert-butyl ether, di ethyl ether, t-butyl methyl ether, diisopropyl ether or mixtures thereof. Finally, lyophilization was carried out to get pure Semaglutide.

The present invention provides a solid phase peptide process for the preparation of Gln(Trt)-Ala-Ala-Lys(C18diacidmono-t-butyl-γ-Glu(AEEA-AEEA)-OtBu)-Glu(OtBu)-Phe-Ile-Ala-Trp (Boc)-Leu-Val-Arg(pbf)-Gly-Arg(pbf)-Gly-OtBu (Fragment-6)

Which comprises:
a) anchoring Fmoc-Arg(Pbf)-OH to a resin in presence of a base;
b) selective deprotection of amino acid using a base;
c) coupling of Fmoc-Gly-OH to a resin obtained in step-b) in presence of coupling agent in a solvent to obtain dipeptide resin;
d) sequential coupling of Fmoc-Arg(Pbf)-OH, Fmoc-Val-OH, Fmoc-Leu-OH, Fmoc-Trp(Boc)-OH, Fmoc-Ala-OH, Fmoc-Ile-OH, Fmoc-Phe-OH, Fmoc-Glu(OtBu)-OH, Fmoc-Lys(C18diacidmono-t-butyl-γ-Glu(AEEA-AEEA)-OtBu)-OH, Fmoc-Ala-OH, Fmoc-Ala-OH, Fmoc-Gln(Trt)-OH to the obtained resin in step-c) in presence of a coupling agent;
e) partial deprotection of peptide obtained in step-d) in presence of a reagent to obtain 14 amino acid chain peptide;
f) coupling of H-Gly-OtBu·HCl to 14 amino acid chain peptide obtained from step-e) in presence of coupling agent;
g) deprotection of protected 15 amino acid peptide chain in step-f) in presence of reagent to obtain fragment-6.

In step-a), CTC resin was taken in a SPPS reactor and dichloromethane was added to it. Fmoc-Arg(Pbf)-OH was added to the resulting reaction mixture in presence of diisopropyl ethylamine.

In step-b), deprotecting the Fmoc group in presence of a base, preferably using 20% piperidine in dimethylformamide.

The reaction temperature may range from 25° C. to 30° C.

In step-c), condensation of peptide resin obtained in step-b) with Fmoc-Gly-OH in presence of coupling agent.

In step-d), sequential addition of Fmoc-Arg(Pbf)-OH, Fmoc-Val-OH, Fmoc-Leu-OH, Fmoc-Trp(Boc)-OH, Fmoc-Ala-OH, Fmoc-Ile-OH, Fmoc-Phe-OH, Fmoc-Glu(OtBu)-OH, Fmoc-Lys[C18diacidmono-t-butyl-γ-Glu(AEEA-AEEA)-OtBu]-OH, Fmoc-Ala-OH, Fmoc-Ala-OH, Fmoc-Gln(Trt)-OH to the obtained resin in step-c) in presence of a coupling agent.

The coupling agent used in this step is using DIC, oxyma pure in DMF.

The reaction temperature may range from 25° C. to 30° C. The duration of the reaction may range from 1 to 4 hours, preferably for the period of 2 to 3 hours.

Deprotection carried out using 20% of piperidine in Dimethyl formamide.

In step-e), partial deprotection is carried out for protected peptide from solid support resin using a reagent to obtain 14 amino acid peptide.

Reagent used in partial deprotection is selected from the group consisting of TFA, TIPS, Water, DTT, Thioanisole, EDT, DMS, cresol, phenol, thiocresol, ammonium iodide, 2,2′-(ethylene dioxy)diethane or its mixture. Preferably using TFA in dichloromethane.

In step-f) coupling of H-Gly-OtBu·HCl to the 14 amino acid peptide chain obtained in step-e) in presence of coupling agent.

The coupling agent used in this step is using EDC·HCl, HOAt in DMF.

In step-g) deprotection of protected 15 amino acid peptide chain in step-f) is carried out in presence of tert-butyl amine.

The present invention provides an alternative solid phase peptide process for the preparation of Gln(Trt)-Ala-Ala-Lys(C18diacidmono-t-butyl-γ-Glu(AEEA-AEEA)-OtBu)-Glu(OtBu)-Phe-Ile-Ala-Trp (Boc)-Leu-Val-Arg(pbf)-Gly-Arg(pbf)-Gly-OtBu (Fragment-6)

Which comprises:
a) anchoring Fmoc-Arg(Pbf)-OH to a resin in presence of a base;
b) selective deprotection of amino acid using a base;
c) coupling of Fmoc-Gly-OH to a resin obtained in step-b) in presence of coupling agent in a solvent to obtain dipeptide resin;
d) sequential coupling of Fmoc-Arg(Pbf)-OH, Fmoc-Val-OH, Fmoc-Leu-OH, Fmoc-Trp(Boc)-OH, Fmoc-Ala-OH, Fmoc-Ile-OH, Fmoc-Phe-OH, Fmoc-Glu(OtBu)-OH to the obtained resin in step-c) in presence of a coupling agent;
e) deprotection of protected peptide obtained in step-d) in presence of reagent to obtain Fmoc-Glu(OtBu)-Phe-Ile-Ala-Trp(Boc)-Leu-Val-Arg(Pbf)-Gly-Arg(Pbf)-OH;
f) coupling of H-Gly-OtBu·HCl to the peptide obtained in step-e) in presence of coupling agent to obtain 11 amino acid chain peptide;
g) coupling of Fmoc-Gln(Trt)-Ala-Ala-Lys(C18diacidmono-t-butyl-γ-Glu(AEEA-AEEA)-OtBu)-OH to the obtained 11 amino acid chain peptide in step-f) in presence of a coupling agent to obtain 15 amino acid chain peptide;
h) partial deprotection of peptide obtained in step-g) in presence of a reagent to obtain fragment-6.

In step-a), CTC resin was taken in a SPPS reactor and dichloromethane was added to it. Fmoc-Arg(Pbf)-OH was added to the resulting reaction mixture in presence of diisopropyl ethylamine.

In step-b), Deprotecting the Fmoc group in presence of a base, preferably using 20% piperidine in dimethylformamide.

The reaction temperature may range from 25° C. to 30° C.

In step-c), condensation of peptide resin obtained in step-b) with Fmoc-Gly-OH in presence of coupling agent.

In step-d), sequential addition of Fmoc-Arg(Pbf)-OH, Fmoc-Val-OH, Fmoc-Leu-OH, Fmoc-Trp(Boc)-OH, Fmoc-Ala-OH, Fmoc-Ile-OH, Fmoc-Phe-OH, Fmoc-Glu(OtBu)-OH to the obtained resin in step-c) in presence of a coupling agent.

The coupling agent used in this step is using DIC, oxyma pure in DMF.

The reaction temperature may range from 25° C. to 30° C. The duration of the reaction may range from 1 to 4 hours, preferably for the period of 2 to 3 hours.

Deprotection carried out using 20% of piperidine in Dimethyl formamide.

In step-e), partial deprotection is carried out for protected peptide from solid support resin using a reagent to obtain Fmoc-Glu(OtBu)-Phe-Ile-Ala-Trp(Boc)-Leu-Val-Arg(Pbf)-Gly-Arg(Pbf)-OH.

Reagent used in partial deprotection is selected from the group consisting of TFA, TIPS, Water, DTT, Thioanisole, EDT, DMS, cresol, phenol, thiocresol, ammonium iodide, 2,2′-(ethylene dioxy)diethane or its mixture. Preferably using TFA in dichloromethane.

In step-f), coupling of H-Gly-OtBu·HCl to the 10 amino acid peptide chain obtained in step-e) in presence of coupling agent.

The coupling agent used in this step is using EDC·HCl, HOAt in DMF.

In step-g), coupling of Fmoc-Gln(Trt)-Ala-Ala-Lys(C18diacidmono-t-butyl-γ-Glu(AEEA-AEEA)-OtBu)-OH to the obtained 11 amino acid chain peptide in step-f) in presence of a coupling agent to obtain 15 amino acid chain peptide.

The coupling agent used in this step is using EDC·HCl, HOAt in DMF.

In step-h), deprotection of protected 15 amino acid peptide chain in step-g) is carried out in presence of tert-butyl amine to obtain fragment-6.

The present invention provides a solid phase peptide process for the preparation of Boc-His(X)-Aib-Glu(OtBu)-Gly-Thr(tBu)-Phe-Thr(tBu)-Ser(tBu)-Asp(OtBu)-Val-Ser(Oxa)-OH (Fragment-7); wherein X is Boc or Trt

which comprises:
a) anchoring Fmoc-Val-Ser(Oxa)-OH to a resin in presence of a base;
b) selective deprotection of amino acid using a base;
c) coupling of Fmoc-Asp(OtBu)-OH to a resin obtained in step-b) in presence of coupling agent in a solvent to obtain dipeptide resin;
d) sequential coupling of Fmoc-Ser(tBu)-OH, Fmoc-Thr(tBu)-OH, Fmoc-Phe-OH, Fmoc-Thr(tBu)-OH, Fmoc-Gly-OH, Fmoc-Glu(OtBu)-OH, Fmoc-Aib-OH, Boc-His(X)-OH to the obtained resin in step-c) in presence of a coupling agent;
e) cleaving of protected peptide from solid support resin in presence of a reagent to get fragment-7.

In step-a), CTC resin was taken in a SPPS reactor and dichloromethane was added to it. Fmoc-Val-Ser(Oxa)-OH was added to the resulting reaction mixture in presence of diisopropyl ethylamine.

In step-b), deprotecting the Fmoc group in presence of a base, preferably using 20% piperidine in dimethylformamide.

The reaction temperature may range from 25° C. to 30° C. The duration of the reaction may range from 1 to 4 hours, preferably for the period of 2-3 hours.

In step-c), condensation of peptide resin obtained in step-b) with Fmoc-Ser(tBu)-OH in presence of coupling agent.

In step-d), sequential addition of Fmoc-Ser(tBu)-OH, Fmoc-Thr(tBu)-OH, Fmoc-Phe-OH, Fmoc-Thr(tBu)-OH, Fmoc-Gly-OH, Fmoc-Glu(OtBu)-OH, Fmoc-Aib-OH, Boc-His(X)-OH (wherein X is Boc or Trt) to the obtained resin in step-c) in presence of a coupling agent.

The coupling agent used in this step is using DIC, oxyma pure in DMF.

The reaction temperature may range from 25° C. to 30° C. The duration of the reaction may range from 1 to 4 hours, preferably for the period of 2 to 3 hours.

Deprotection carried out using 20% of piperidine in Dimethylformamide.

In step-e) cleavage is carried out for protected peptide from solid support resin using a reagent to obtain Fragment-7.

Reagent used in cleavage step is selected from the group consisting of TFA, TIPS, Water, DTT, Thioanisole, EDT, DMS, cresol, phenol, thiocresol, ammonium iodide, 2,2′-(ethylene dioxy)diethane or its mixture. Preferably using TFA in dichloromethane.

Accordingly, the present invention provides solution phase peptide process for the preparation of Semaglutide of compound of formula-I by using three fragment approach.

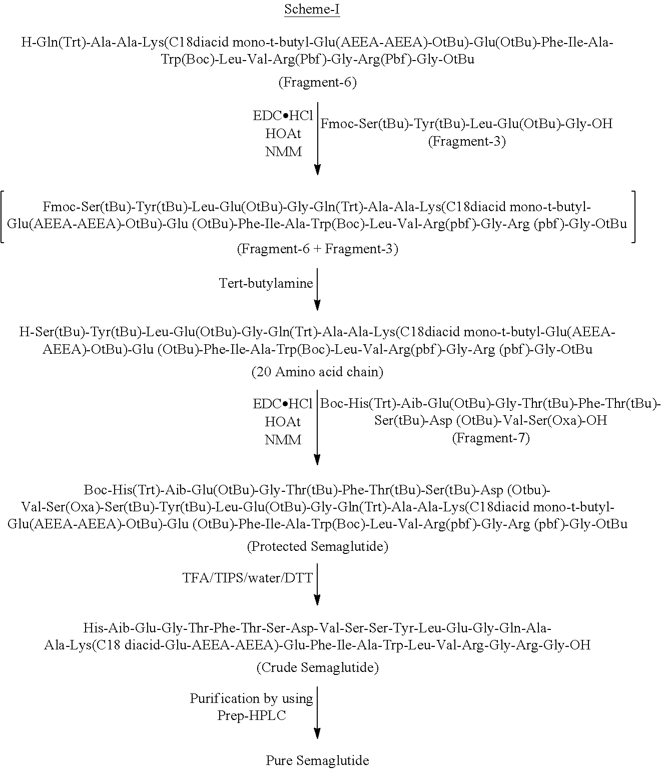

Fragments-3, -6 and -7 are prepared by using solid phase peptide synthesis.

In step-a), Gln(Trt)-Ala-Ala-Lys(C18diacidmono-t-butyl-γ-Glu(AEEA-AEEA)-OtBu)-Glu (OtBu)-Phe-Ile-Ala-Trp(Boc)-Leu-Val-Arg(pbf)-Gly-Arg(pbf)-Gly-OtBu (Fragment-6) was condensed with Fmoc-Ser(tBu)-Tyr(tBu)-Leu-Glu(OtBu)-Gly-OH (Fragment-3) in presence of coupling agent to obtain Fmoc-Ser(tBu)-Tyr(tBu)-Leu-Glu(OtBu)-Gly-Gln(Trt)-Ala-Ala-Lys (C18diacidmono-t-butyl-γ-Glu(AEEA-AEEA)-OtBu)-Glu(OtBu)-Phe-Ile-Ala-Trp(Boc)-Leu-Val-Arg(pbf)-Gly-Arg (pbf)-Gly-OtBu in in-situ manner. Further, it is deprotected in presence of a base to obtain Ser(tBu)-Tyr(tBu)-Leu-Glu(OtBu)-Gly-Gln(Trt)-Ala-Ala-Lys(C18 diacid mono-t-butyl-γ-Glu(AEEA-AEEA)-OtBu)-Glu(OtBu)-Phe-Ile-Ala-Trp(Boc)-Leu-Val-Arg(pbf)-Gly-Arg (pbf)-Gly-OtBu.

The coupling agent used in the reaction can be selected from group consisting of [Ethylcyano (hydroxyimino)acetate)-tri-(1-pyrrolidinyl)-Phosphonium hexa fluorophosphate (PyOxim), ethyl-2-cyano-2-(hydroxy amino) acetate (Oxyma pure), O-(benzotriazol-1-yl)-N,N,N′,N′-tetramethyluronium tetrafluoroborate (TBTU), diisopropyl carbodiimide (DIC), 1,3-dicyclohexylcabodiimide (DCC), O-(7-azabenzotriazol-1-yl)-N,N,N′,N′-tetramethyluronium hexafluorophosphate (HATU), 1-(dimethyl aminopropyl)-3-ethylcarbodiimide hydrochloride (EDC HCl), O-(benzotriazol-1-yl)-1,1,3,3-tetra methyluronium hexafluorophosphate (HBTU), 1-Hydroxybenzotriazole (HOBt), 1-Hydroxy-7-azabenzotriazole (HOAt), Isopropyl chloro formate (IPCF), Benzotriazol-1-yl-oxy-tris(dimethyl-amino)-phosphonium hexa fluorophosphate (BOP), benzotriazole-1-yloxytri(pyrrolidino)phosphonium hexa fluoro phosphate (PyBOP), N,N-bis-(2-oxo-3-oxazolidinyl)phosphonic dichloride (BOP-Cl), bromotri(pyrrolidino)phosphonium hexa fluoro phosphate (PyBrOP), O-(6-Chloro-1-hydrocibenzotriazol-1-yl)-1,1,3,3-tetramethyl uranium tetra fluoroborate (TCTU), chlorotri (pyrrolidino)phosphonium hexafluorophosphate (PyClOP), Ethyl 1,2-dihydro-2-ethoxyquinoline-carboxylate (EEDQ), isobutyl chloro formate (IBCF), 2-succinimido-1,1,3,3-tetramethyluronium tetrafluoroborate (TSTU), 1-Cyano-2-ethoxy-2-oxo ethylidene aminooxy) dimethyl amino morpholino-carbeniumhexafluorophosphate (COMU), 2-(5-norbornen-2,3-dicarboximido)-1,1,3,3-tetramethyluronium tetrafluoroborate (TNTU), propane phosphonic acid anhydride (PPAA), 3-(diethoxy phosphoryloxy)-1,2,3-benzotriazin-4(3H)-one (DEPBT) or its mixture. Preferably using EDC·HCl and HOAt in DCM.

The reaction temperature may range from 25° C. to 30° C. The duration of the reaction may range from 4 to 8 hours, preferably for the period of 6 to 7 hours.

Deprotection of Fmoc-Ser(tBu)-Tyr(tBu)-Leu-Glu(OtBu)-Gly-Gln(Trt)-Ala-Ala-Lys(C18 diacid mono-t-butyl-γ-Glu(AEEA-AEEA)-OtBu)-Glu(OtBu)-Phe-Ile-Ala-Trp(Boc)-Leu-Val-Arg(pbf)-Gly-Arg(pbf)-Gly-OtBu was carried out by using abase in in-situ manner. The base used in the reaction can be selected from group consisting of tert-butyl amine, 20% of 4-methyl piperidine in Dimethyl formamide, 20% of piperidine in Dimethyl formamide and 20% of piperazine in Dimethyl formamide. Preferably using tert-butylamine.

In step-b), Boc-His(X)-Aib-Glu(OtBu)-Gly-Thr(tBu)-Phe-Thr(tBu)-Ser(tBu)-Asp(OtBu)-Val-Ser(Oxa)-OH (fragment-7); where in X is Boc or Trt was condensed with peptide obtained from step-a) in presence of a coupling agent to obtain protected Semaglutide.

Coupling agent used in the reaction is preferably EDC·HCl and HOAt in DMF.

The reaction temperature may range from 25° C. to 30° C. The duration of the reaction may range from 1 to 3 hours, preferably for the period of 1 to hours.

In step-c), protected Semaglutide obtained from step-b) was deprotected using a reagent to obtain crude Semaglutide.

Reagent used in cleavage step is selected from the group consisting of TFA, TIPS, Water, DTT, Thioanisole, EDT, DMS, cresol, phenol, thiocresol, ammonium iodide, 2,2′-(ethylene dioxy)diethane or its mixture. Preferably using cocktail mixture of TFA, TIPS, water and DTT.

The deprotection of protected peptide carried out by treating with an acid and at least one scavenger. The peptide cleavage reagent used in the process of the present invention is a cocktail mixture of acid, scavengers and solvents.

The reaction temperature may range from 5° C. to 30° C., preferably 10-15° C. The duration of the reaction may range from 2 to 6 hours, preferably for the period of 3-4 hours.

In step-d), the obtained crude Semaglutide was purified on reverse phase HPLC using a buffer and a solvent, followed by freeze drying to obtain Semaglutide.

Where, the buffer used in the reaction is selected from the group consisting of Glacial acetic acid, ammonia solution, Trifluoroacetic anhydride in water, Purified water, Orth phosphoric acid in water, acetonitrile, Triton-X-100, ethanol, methanol, ethyl acetate, triethyl amine in water, ammonium acetate in water, ammonium bicarbonate in water or its mixture.

The Fmoc protected amino acids and Fmoc-Lys(C18diacidmono-t-butyl-γ-Glu(AEEA-AEEA)-OtBu)-OH are commercially available or may be prepared according to procedures known in the prior art literature or it may be prepared by the process disclosed in IN 202141019461.

The coupling reactions may be monitored by Kaiser test or TNBS test or chloranil test.

The cleavage of the peptide from the solid support may be accomplished by any conventional methods well known in the art.

Accordingly, the present invention provides solution phase peptide process for the preparation of Semaglutide of compound of formula-I by using four fragment approach.

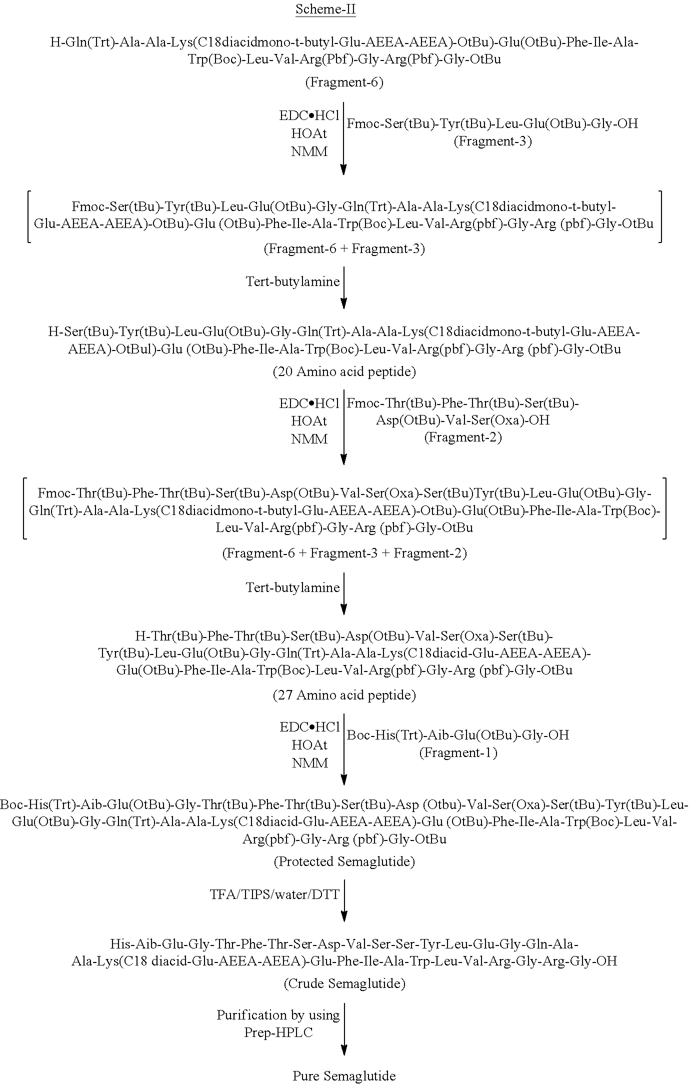

The present invention provides a hybrid approach for the preparation of Semaglutide compound of formula-I.

Wherein, Fragments-1, -2, -3 and -6 are prepared by using solid phase peptide synthesis. In step-a), H-Gln(Trt)-Ala-Ala-Lys(C18diacidmono-t-butyl-γ-Glu(AEEA-AEEA)-OtBu)-Glu (OtBu)-Phe-Ile-Ala-Trp(Boc)-Leu-Val-Arg(pbf)-Gly-Arg(pbf)-Gly-OtBu (fragment-6) was condensed with Fmoc-Ser(tBu)-Tyr(tBu)-Leu-Glu(OtBu)-Gly-OH (fragment-3) in presence of coupling agent obtain Fmoc-Ser(tBu)-Tyr(tBu)-Leu-Glu(OtBu)-Gly-Gln(Trt)-Ala-Ala-Lys(C18 diacidmono-t-butyl-γ-Glu(AEEA-AEEA)-OtBu)-Glu(OtBu)-Phe-Ile-Ala-Trp(Boc)-Leu-Val-Arg(pbf)-Gly-Arg(pbf)-Gly-OtBu in in-situ manner. Further, it is deprotected in presence of a base to obtain Ser(tBu)-Tyr(tBu)-Leu-Glu(OtBu)-Gly-Gln(Trt)-Ala-Ala-Lys(C18-di acid mono-t-butyl-γ-Glu(AEEA-AEEA)-OtBu)-Glu(OtBu)-Phe-Ile-Ala-Trp(Boc)-Leu-Val-Arg(pbf)-Gly-Arg (pbf)-Gly-OtBu.

The coupling agent used in the reaction can be selected from group consisting of [Ethylcyano (hydroxyimino)acetate)-tri-(1-pyrrolidinyl)-Phosphonium hexafluorophosphate (PyOxim), ethyl-2-cyano-2-(hydroxyamino)acetate (Oxyma pure), O-(benzotriazol-1-yl)-N,N,N′,N′-tetramethyl uronium tetrafluoroborate (TBTU), diisopropyl carbodiimide (DIC), 1,3-dicyclohexyl carbodiimide (DCC), O-(7-azabenzotriazol-1-yl)-N,N,N′,N′-tetramethyluronium hexafluoro phosphate (HATU), 1-(dimethyl aminopropyl)-3-ethylcarbodiimide hydrochloride (EDC HCl), O-(benzotriazol-1-yl)-1,1,3,3-tetra methyluronium hexafluorophosphate (HBTU), 1-Hydroxy benzotriazole (HOBt), 1-Hydroxy-7-azabenzotriazole (HOAt), Isopropyl chloro formate (IPCF), Benzotriazol-1-yl-oxy-tris(dimethyl-amino)-phosphonium hexa fluorophosphate (BOP), benzotriazole-1-yloxytri(pyrrolidino)phosphonium hexa fluoro phosphate (PyBOP), N,N-bis-(2-oxo-3-oxazolidinyl)phosphonic dichloride (BOP-Cl), bromotri(pyrrolidino)phosphonium hexa fluorophosphate (PyBrOP), O-(6-Chloro-1-hydrocibenzotriazol-1-yl)-1,1,3,3-tetramethyl uranium tetra fluoroborate (TCTU), chlorotri(pyrrolidino)phosphonium hexafluorophosphate (PyClOP), Ethyl 1,2-dihydro-2-ethoxyquinoline-carboxylate (EEDQ), isobutyl chloro formate (IBCF), 2-succinimido-1,1,3,3-tetramethyluronium tetrafluoroborate (TSTU), 1-Cyano-2-ethoxy-2-oxo ethylidene aminooxy) dimethyl amino morpholino-carbeniumhexafluorophosphate (COMU), 2-(5-norbornen-2,3-dicarboximido)-1,1,3,3-tetramethyluronium tetrafluoroborate (TNTU), propanephosphonic acid anhydride (PPAA), 3-(diethoxy phosphoryloxy)-1,2,3-benzotriazin-4(3H)-one (DEPBT) or its mixture. Preferably using EDC·HCl and HOAt in DMF.

The reaction temperature may range from 25° C. to 30° C. The duration of the reaction may range from 1 to 3 hours, preferably for the period of 1 to 2 hours.

Deprotection of Fmoc-Ser(tBu)-Tyr(tBu)-Leu-Glu(OtBu)-Gly-Gln(Trt)-Ala-Ala-Lys(C18 diacid mono-t-butyl-γ-Glu(AEEA-AEEA)-OtBu)-Glu(OtBu)-Phe-Ile-Ala-Trp(Boc)-Leu-Val-Arg(pbf)-Gly-Arg(pbf)-Gly-OtBu was carried out by using abase in in-situ manner. The base used in the reaction can be selected from group consisting of tert-butyl amine, 20% of 4-methyl piperidine in Dimethyl formamide, 20% of piperidine in Dimethyl formamide and 20% of piperazine in Dimethyl formamide. Preferably using tert-butylamine.

In step-b), Fmoc-Thr(tBu)-Phe-Thr(tBu)-Ser(tBu)-Asp(OtBu)-Val-Ser(Oxa)-OH (fragment-2) was condensed with peptide obtained from step-a) in presence of a coupling agent to obtain Fmoc protected Thr(tBu)-Phe-Thr(tBu)-Ser(tBu)-Asp(OtBu)-Val-Ser(Oxa)-Ser(tBu)-Tyr(tBu)-Leu-Glu(OtBu)-Gly-Gln(Trt)-Ala-Ala-Lys(C18diacidmono-t-butyl-γ-Glu(AEEA-AEEA)-OtBu)-Glu (OtBu)-Phe-Ile-Ala-Trp(Boc)-Leu-Val-Arg(pbf)-Gly-Arg (pbf)-Gly-OtBu in in-situ manner.

Coupling agent used in the reaction is preferably EDC·HCl and HOAt in DMF. The reaction temperature may range from 25° C. to 30° C. The duration of the reaction may range from 1 to 3 hours, preferably for the period of 1 to 2 hours.

Deprotection of Fmoc-Thr(tBu)-Phe-Thr(tBu)-Ser(tBu)-Asp(OtBu)-Val-Ser(Oxa)-Ser(tBu)-Tyr (tBu)-Leu-Glu(OtBu)-Gly-Gln(Trt)-Ala-Ala-Lys(C18diacidmono-t-butyl-γ-Glu(AEEA-AEEA)-OtBu)-Glu(OtBu)-Phe-Ile-Ala-Trp(Boc)-Leu-Val-Arg(pbf)-Gly-Arg(pbf)-Gly-OtBu was carried out by using a base in in-situ manner. The base used in the reaction can be selected from group consisting of tert-butyl amine, 20% of 4-methyl piperidine in Dimethyl formamide, 20% of piperidine in Dimethyl formamide and 20% of piperazine in Dimethyl formamide. Preferably using tert-butylamine.

In step-c), Boc-His(X)-Aib-Glu(OtBu)-Gly-OH (fragment-1); wherein X is Boc or Trt was condensed with the peptide obtained in step-b) in presence of a coupling agent and solvent to obtain protected Semaglutide.

Coupling agent used in the reaction is preferably EDC·HCl and HOAt in DMF. The reaction temperature may range from 25° C. to 30° C. The duration of the reaction may range from 1 to 3 hours, preferably for the period of 1 to 2 hours.

In step-d) protected Semaglutide obtained from step-c) was deprotected using a reagent to obtain crude Semaglutide.

Reagent used in cleavage step is selected from the group consisting of TFA, TIPS, Water, DTT, Thioanisole, EDT, DMS, cresol, phenol, thiocresol, ammonium iodide, 2,2′-(ethylene dioxy)diethane or its mixture. Preferably using cocktail mixture of TFA, TIPS, water and DTT.

The deprotection of protected peptide carried out by treating with an acid and at least one scavenger. The peptide cleavage reagent used in the process of the present invention is a cocktail mixture of acid, scavengers and solvents.

The reaction temperature may range from 5° C. to 30° C., preferably 10-15° C. The duration of the reaction may range from 2 to 6 hours, preferably for the period of 3-4 hours.

In step-e), the obtained crude Semaglutide was purified on reverse phase HPLC using a buffer and a solvent, followed by freeze drying to obtain Semaglutide.

Accordingly, the present invention provides solution phase peptide process for the preparation of Semaglutide of compound of formula-I by using five fragment approach.

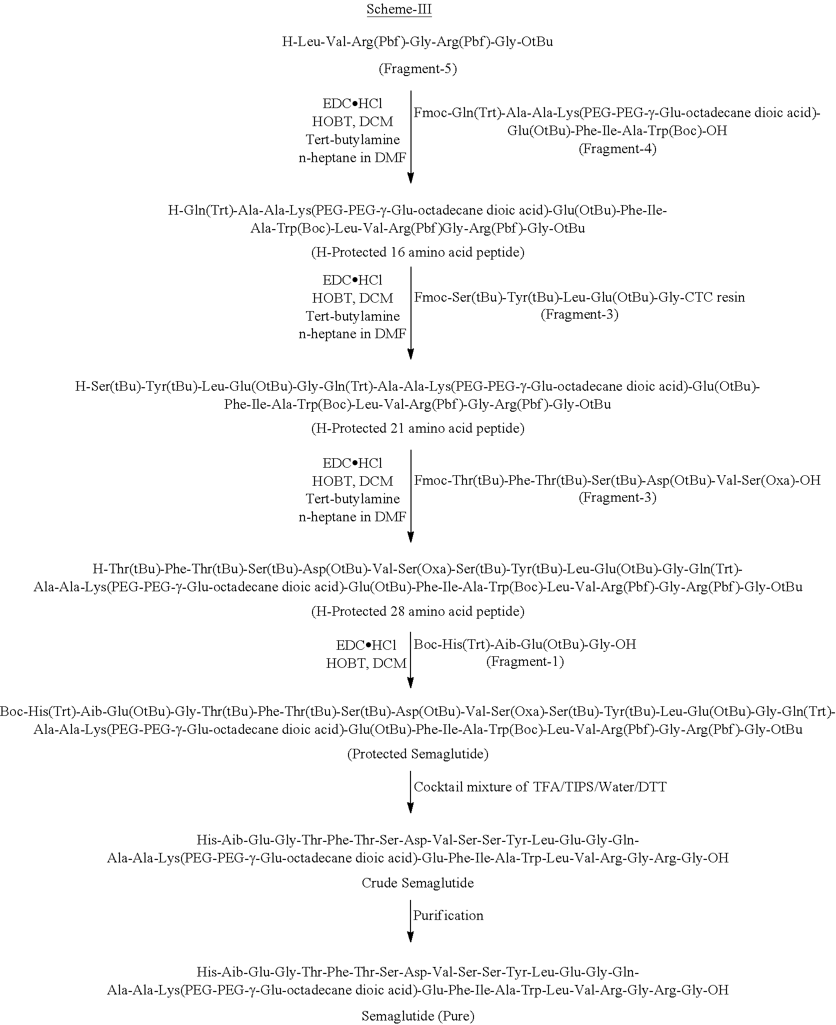

In step-a), Fmoc-Gln(Trt)-Ala-Ala-Lys(C18 diacid mono-t-butyl-γ-Glu(AEEA-AEEA)-OtBu)-Glu(OtBu)-Phe-Ile-Ala-Trp(Boc)-OH (fragment-4) was condensed with Leu-Val-Arg(pbf)-Gly-Arg(pbf)-Gly-OtBu (fragment-5) in presence of coupling agent to obtain protected Gln(Trt)-Ala-Ala-Lys(C18 diacid mono-t-butyl-γ-Glu(AEEA-AEEA)-OtBu)-Glu(OtBu)-Phe-Ile-Ala-Trp(Boc)-Leu-Val-Arg (pbf)-Gly-Arg (pbf)-Gly-OtBu.

The coupling agent used in the reaction can be selected from group consisting of [Ethylcyano (hydroxyimino)acetate-02)-tri-(1-pyrrolidinyl)-Phosphonium hexa fluorophosphate (PyOxim), ethyl-2-cyano-2-(hydroxy amino) acetate (Oxyma pure), O-(benzotriazol-1-yl)-N,N,N′,N′-tetramethyluronium tetrafluoroborate (TBTU), diisopropyl carbodiimide (DIC), 1,3-dicyclohexylcabodiimide (DCC), O-(7-azabenzotriazol-1-yl)-N,N,N′,N′-tetramethyluronium hexafluorophosphate (HATU), 1-(dimethyl aminopropyl)-3-ethylcarbodiimide hydrochloride (EDC HCl), O-(benzotriazol-1-yl)-1,1,3,3-tetra methyluronium hexafluorophosphate (HBTU), 1-Hydroxybenzotriazole (HOBt), Isopropyl chloro formate (IPCF), Benzotriazol-1-yl-oxy-tris(dimethyl-amino)-phosphonium hexa fluorophosphate (BOP), benzotriazole-1-yloxytri(pyrrolidino)phosphonium hexa fluoro phosphate (PyBOP), N,N-bis-(2-oxo-3-oxazolidinyl)phosphonic dichloride (BOP-Cl), bromotri(pyrrolidino)phosphonium hexa fluoro phosphate (PyBrOP), O-(6-Chloro-1-hydrocibenzotriazol-1-yl)-1,1,3,3-tetramethyl uranium tetra fluoroborate (TCTU), chlorotri (pyrrolidino)phosphonium hexafluorophosphate (PyClOP), Ethyl 1,2-dihydro-2-ethoxyquinoline-carboxylate (EEDQ), isobutyl chloro formate (IBCF), 2-succinimido-1,1,3,3-tetramethyluronium tetrafluoroborate (TSTU), 1-Cyano-2-ethoxy-2-oxo ethylidene aminooxy) dimethyl amino morpholino-carbeniumhexafluorophosphate (COMU), 2-(5-norbornen-2,3-dicarboximido)-1,1,3,3-tetramethyluronium tetrafluoroborate (TNTU), propane phosphonic acid anhydride (PPAA), 3-(diethoxy phosphoryloxy)-1,2,3-benzotriazin-4(3H)-one (DEPBT) or its mixture. Preferably using EDC·HCl and HOBt in DCM.

The reaction temperature may range from 25° C. to 30° C. The duration of the reaction may range from 10 to 30 minutes, preferably for the period of 15 to 20 minutes.

Deprotection of Fmoc-Gln(Trt)-Ala-Ala-Lys(C18 diacid mono-t-butyl-γ-Glu(AEEA-AEEA)-OtBu)-Glu(OtBu)-Phe-Ile-Ala-Trp(Boc)-Leu-Val-Arg(pbf)-Gly-Arg(pbf)-Gly-OtBu was carried out by using a base. The base used in the reaction can be selected from group consisting of tert-butyl amine, 20% of 4-methyl piperidine in Dimethylformamide, 20% of piperidine in Dimethyl formamide and 20% of piperazine in Dimethylformamide. Preferably using tert-butyl amine.

In step-b), Fmoc-Ser(tBu)-Tyr(tBu)-Leu-Glu(OtBu)-Gly-OH (fragment-3) was condensed with peptide obtained from step-a) in presence of a coupling agent to obtain Fmoc-Ser(tBu)-Tyr(tBu)-Leu-Glu(OtBu)-Gly-Gln(Trt)-Ala-Ala-Lys(C18 diacid mono-t-butyl-γ-Glu(AEEA-AEEA)-OtBu)-Glu(OtBu)-Phe-Ile-Ala-Trp(Boc)-Leu-Val-Arg(pbf)-Gly-Arg (pbf)-Gly-OtBu.

Coupling agent used in the reaction is preferably EDC·HCl and HOBt in DCM. The reaction temperature may range from 25° C. to 30° C. The duration of the reaction may range from 10 to 30 minutes, preferably for the period of 15 to 20 minutes. Deprotection of peptide was carried out by using a base. The base used in the reaction is tert-butyl amine.

In step-c), Fmoc-Thr(tBu)-Phe-Thr(tBu)-Ser(tBu)-Asp(OtBu)-Val-Ser(Oxa)-OH (fragment-2) was condensed with peptide obtained from step-b) in presence of a coupling agent to obtain Thr(tBu)-Phe-Thr(tBu)-Ser(tBu)-Asp(OtBu)-Val-Ser(Oxa)-Ser(tBu)-Tyr(tBu)-Leu-Glu(OtBu)-Gly-Gln(Trt)-Ala-Ala-Lys(C18 diacid mono-t-butyl-γ-Glu(AEEA-AEEA)-OtBu)-Glu(OtBu)-Phe-Ile-Ala-Trp (Boc)-Leu-Val-Arg (pbf) Gly-Arg (pbf)-Gly-OtBu peptide.

Coupling agent used in the reaction is EDC·HCl and HOBt in DCM. The reaction temperature may range from 25° C. to 30° C. The duration of the reaction may range from 10 to 30 minutes, preferably for the period of 15 to 20 minutes.

Deprotection of obtained peptide was carried out by using a base. The base used in the reaction is tert-butyl amine.

In step-d), Boc-His(Trt)-Aib-Glu(OtBu)-Gly-OH (fragment-1) was condensed with peptide obtained in step-c) in presence of a coupling agent to obtain protected Semaglutide.

Coupling agent used in the reaction is EDC·HCl and HOBt in DCM. The reaction temperature may range from 25° C. to 30° C. The duration of the reaction may range from 10 to 30 minutes, preferably for the period of 15 to 20 minutes.

In step-e), protected Semaglutide obtained from step-d) was deprotected using a reagent to obtain crude Semaglutide. Reagent used in cleavage step is selected from the group consisting of TFA, TIPS, Water, DTT, Thioanisole, EDT, DMS, cresol, phenol, thiocresol, ammonium iodide, 2,2′-(ethylene dioxy)diethane or its mixture. Preferably using cocktail mixture of TFA, TIPS, water and DTT.

The deprotection of protected peptide carried out by treating with an acid and at least one scavenger. The peptide cleavage reagent used in the process of the present invention is a cocktail mixture of acid, scavengers and solvents.

The reaction temperature may range from 5° C. to 30° C., preferably 10 to 15° C. The duration of the reaction may range from 2 to 6 hours, preferably for the period of 3 to 4 hours.

In step-g), the obtained crude Semaglutide was purified on reverse phase HPLC using a buffer and a solvent, followed by freeze drying to obtain Semaglutide.

where the buffer used in the reaction is selected from the group consisting of Glacial acetic acid, ammonia solution, Trifluoroacetic anhydride in water, Purified water, Ortho phosphoric acid in water, acetonitrile, Triton-X-100, ethanol, methanol, ethyl acetate, triethyl amine in water, ammonium acetate in water, ammonium bicarbonate in water or its mixture.

The Fmoc protected amino acids are commercially available or may be prepared according to procedures known in the literature.

The coupling reactions may be monitored by Kaiser test. The cleavage of the peptide from the solid support may be accomplished by any conventional methods well known in the art.

The present invention also provides a solid phase peptide process for the preparation of Fmoc-Gln(Trt)-Ala-Ala-Lys(C18 diacid mono-t-butyl-γ-Glu(AEEA-AEEA)-OtBu)-Glu(OtBu)-Phe-Ile-Ala-Trp (Boc)-OH of fragment-4

which comprises:
a) anchoring Fmoc-Trp(Boc)-OH to a resin in presence of a coupling agent;
b) selective deprotection of amino acid using a base;
c) coupling of Fmoc-Ala-OH to a resin obtained in step-b) in presence of coupling agent in a solvent to obtain dipeptide resin;
d) sequential coupling of Fmoc-Ile-OH, Fmoc-Phe-OH, Fmoc-Glu(OtBu)-OH, Fmoc-Lys(C18 diacid mono-t-butyl-γ-Glu(AEEA-AEEA)-OtBu)-OH, Fmoc-Ala-OH, Fmoc-Ala-OH, Fmoc-Gln(Trt)-OH to the obtained resin in step-c) in presence of a coupling agent;
e) cleaving of protected peptide from solid support resin in presence of a reagent to get fragment-4.

In step-a), CTC resin was taken in a SPPS reactor and dichloromethane was added to it. Deprotecting the Fmoc group in presence of a base, preferably using 20% piperidine in dimethylformamide.

In step-c), condensation of peptide resin obtained in step-b) with Fmoc-Ala-OH in presence of a coupling agent. The coupling agent used in the reaction is DIC oxyma pure in DMF. The reaction temperature may range from 25° C. to 30° C. The duration of the reaction may range from 1 to 4 hours, preferably for the period of 2-3 hours.

In step-d) Sequential addition of Fmoc-Ile-OH, Fmoc-Phe-OH, Fmoc-Glu(OtBu)-OH, Fmoc-Lys(C18 diacid mono-t-butyl-γ-Glu(AEEA-AEEA)-OtBu)-OH, Fmoc-Ala-OH, Fmoc-Ala-OH, Fmoc-Gln(Trt)-OH to the obtained resin in step-a) in presence of a coupling agent.

The coupling agent used in this step is preferably using DIC, oxyma pure in DMF.

Deprotection carried out using 20% of piperidine in Dimethyl formamide.

In step-e) cleavage is carried out for protected peptide from solid support resin using a reagent to obtain crude Semaglutide.

### Preparative HPLC Method for Purification of Semaglutide:

Trifluoroacetic Acid Purification-1:

Sample preparation: 5 Grams of crude Semaglutide was dissolved in 800 mL of water and 25% aqueous ammonia solution added dropwise to get the clear solution.

### Column: YMC Triart (50×250 mm, 10 μm)

Mobile phase-A: Tri fluoro acetic acid (5 mL)+water (5 mL)
Mobile phase-B: Isopropyl alcohol (2.5 mL)+Acetonitrile (2.5 mL)+Ortho phosphoric acid (5 mL)
Equilibrate the column with 5% mobile phase B at a flow rate of 60 mL/minute.

| | | Flow | Mobile Phase | Mobile Phase |
|---|---|---|---|---|
| S. No | Time | (mL/min) | A % | B % |
| 1 | 0.01 | 60 | 95 | 5 |
| 2 | 10 | 60 | 75 | 25 |
| 3 | 150 | 60 | 40 | 60 |
| 4 | 200 | 60 | 0 | 100 |
| 5 | 300 | 60 | 0 | 100 |

Collect the fractions as 25 mL/vial

Ammonium Bicarbonate Purification-2:

Fraction obtained from the above purification process is diluted with water.
Mobile phase-A: water (5 Ltr)+Ammonium bicarbonate (8.0 gms);
Mobile phase-B: Acetonitrile:water (8:2)
Equilibrate the column with 5% mobile phase-B with a flow rate of 50 mL/min.

| | | Flow | Mobile Phase | Mobile Phase |
|---|---|---|---|---|
| S. No | Time | (mL/min) | A % | B % |
| 1 | 0.01 | 50 | 95 | 5 |
| 2 | 10 | 50 | 75 | 25 |
| 3 | 150 | 50 | 50 | 50 |
| 4 | 200 | 50 | 0 | 100 |
| 5 | 300 | 50 | 0 | 100 |

Collect the fractions as 25 mL/vial and pooled fraction was lyophilized to get the pure Semaglutide.

### Purity: 97.2%

### EXPERIMENTAL PORTION

The details of the invention are given in the examples provided below, which are given to illustrate the invention only and therefore should not be construed to limit the scope of the invention.

### Example-1: Process for the Preparation of Semaglutide by Using Five Fragments Through Hybrid Approach

### Stage-1: Synthesis of Boc-His(Trt)-Aib-Glu(OtBu)-Gly-OH [Fragment-1]

Step-A: CTC resin (50 grams) was taken in a SPPS reactor and dichloromethane (120 mL) was added and allowed it to swell for 10 minutes.

Step-B: A solution of Fmoc-Gly-OH (3 equivalents) and Diisopropylethylamine (5 equivalents) in dry dichloromethane was added to the resin obtained from step-A and stirred for 2 hours at room temperature. The above resin was deblocked with 20% piperidine in DMF for 10-15 minutes and washed with DMF.

Step-C: Fmoc-Glu(OtBu)-OH (82.8 grams) was dissolved in DMF and stirred for 10 minutes. DIC (30.1 grams) and oxyma (27.69 grams) were added to the resulting reaction mixture and stirred for 5-10 minutes at the room temperature. It was added to the resin obtained in step-A and stirred for 2-3 hours at room temperature. The progress of coupling was monitored by Kaiser tests. After completion of the reaction, the resin was drained and washed with DMF, isopropanol and dichloromethane. The resulting resin was deblocked with 20% piperidine in DMF for 10 minutes and washed with DMF.

Step-D: Fmoc-Aib-OH (63.4 grams) was dissolved in DMF and stirred for 10 minutes. DIC (30.1 grams) and oxyma (27.69 grams) were added to the resulting reaction mixture and stirred for 5-10 minutes at the same temperature. It was added to the resin obtained in step-A and stirred for 2-3 hours at room temperature. The progress of coupling was monitored by Kaiser test. After completion of reaction the resin was drained and washed with DMF, isopropanol and dichloromethane. The resulting resin was deblocked with 20% piperidine in DMF.

Step-E: Boc-His(Trt)-OH (96.9 grams) was dissolved in DMF and stirred for 10 minutes. DIC (30.1 grams) and oxyma (27.69 grams) were added to the resulting reaction mixture and stirred for 5-10 minutes at the same temperature. It was added to the resin obtained in step-A and stirred for 2-3 hours at room temperature. The progress of coupling was monitored by Kaiser test. After completion of reaction the resin was washed with DMF, isopropanol and dichloromethane.

Step-F: Selective cleavage of CTC-resin from Boc-His(Trt)-Aib-Glu(OtBu)-Gly-CTC resin was performed with a mixture of 1% Trifluoroacetic acid in dichloromethane. The crude protected peptide was isolated by precipitating with ether.

Yield: 92.5%; Purity: 97.02%

Stage-2: Synthesis of Fmoc-Thr(tBu)-Phe-Thr(tBu)-Ser(tBu)-Asp(OtBu)-Val-Ser(Oxa)-OH [Fragment-2]

Step-A: CTC resin (50 grams) was taken in a SPPS reactor and dichloromethane was added and allowed it to swell for 10 minutes.

Step-B: A solution of Fmoc-Val-Ser(Oxa)-OH (41 grams) and Diisopropylethylamine (20.77 grams) in dry dichloromethane was added to the resin obtained from step-A and stirred for 2 hours at room temperature. The above resin was deblocked with 20% piperidine in DMF for 10-15 minutes and washed with DMF.

Step-C: Fmoc-Ser(tBu)-OH (18.49 grams) was dissolved in DMF and stirred for 10 minutes. DIC (6 grams) and oxyma (6.82 grams) were added to the resulting reaction mixture and stirred for 5-10 minutes at the room temperature. It was added to the resin obtained in step-A and stirred for 2-3 hours at room temperature. The progress of coupling was monitored by Kaiser tests. After completion of the reaction, the resin was drained and washed with DMF, isopropanol and dichloromethane. The resulting resin was deblocked with 20% piperidine in DMF for 10 minutes and washed with DMF.

Step-D: Fmoc-Thr(tBu)-OH (19.08 grams) was dissolved in DMF and stirred for 10 minutes. DIC (6 grams) and oxyma (6.82 grams) were added to the resulting reaction mixture and stirred for 5-10 minutes at the same temperature. It was added to the resin obtained in step-A and stirred for 2-3 hours at room temperature. The progress of coupling was monitored by Kaiser test. After completion of reaction the resin was drained and washed with DMF, isopropanol and dichloromethane. The resulting resin was deblocked with 20% piperidine in DMF.

Step-E: Fmoc-Phe-OH (18.60 grams) was dissolved in DMF and stirred for 10 minutes. DIC (6.0 grams) and oxyma (6.82 grams) were added to the resulting reaction mixture and stirred for 5-10 minutes at the same temperature. It was added to the resin obtained in step-A and stirred for 2-3 hours at room temperature. The progress of coupling was monitored by Kaiser test. After completion of reaction the resin was washed with DMF, isopropanol and dichloromethane.

Step-F: Fmoc-Thr(tBu)-OH (19.08 grams) was dissolved in DMF and stirred for 10 minutes. DIC (6.0 grams) and oxyma (6.82 grams) were added to the resulting reaction mixture and stirred for 5-10 minutes at the same temperature. It was added to the resin obtained in step-A and stirred for 2-3 hours at room temperature. The progress of coupling was monitored by Kaiser test. After completion of reaction the resin was washed with DMF, isopropanol and dichloromethane.

Step-G: Selective cleavage of CTC-resin from Fmoc-Thr(tBu)-Phe-Thr(tBu)-Ser(tBu)-Asp(OtBu)-Val-Ser(Oxa)-CTC resin was performed with a mixture of 1% Trifluoroacetic acid in dichloromethane. The crude protected peptide was isolated by precipitating with ether.

Yield: 99.42%; Purity: 99.42%

Stage-3: Synthesis of Fmoc-Ser(tBu)-Tyr(tBu)-Leu-Glu(OtBu)-Gly-OH [Fragment-3]

Step-A: CTC resin (50 grams) was taken in a SPPS reactor and dichloromethane was added and allowed it to swell for 10 minutes.

Step-B: A solution of Fmoc-Gly-OH (71.4 grams) and Diisopropylethylamine (51.72 grams) in dry dichloromethane was added to the resin obtained from step-A and stirred for 2 hours at room temperature. The above resin was deblocked with 20% piperidine in DMF for 10-15 minutes and washed with DMF.

Step-C: Fmoc-Gly(OtBu)-OH (63.82 grams) was dissolved in DMF and stirred for 10 minutes. DIC (18.92 grams) and oxyma (21.3 grams) were added to the resulting reaction mixture and stirred for 5-10 minutes at the room temperature. It was added to the resin obtained in step-A and stirred for 2-3 hours at room temperature. The progress of coupling was monitored by Kaiser tests. After completion of the reaction, the resin was drained and washed with DMF, isopropanol and dichloromethane. The resulting resin was deblocked with 20% piperidine in DMF for 10 minutes and washed with DMF.

Step-D: Fmoc-Leu-OH (53.01 grams) was dissolved in DMF and stirred for 10 minutes. DIC (18.92 grams) and oxyma (21.30 grams) were added to the resulting reaction mixture and stirred for 5-10 minutes at the same temperature. It was added to the resin obtained in step-A and stirred for 2-3 hours at room temperature. The progress of coupling was monitored by Kaiser test. After completion of reaction the resin was drained and washed with DMF, isopropanol and dichloromethane. The resulting resin was deblocked with 20% piperidine in DMF.

Step-E: Fmoc-Tyr(tBu)-OH (69 grams) was dissolved in DMF and stirred for 10 minutes. DIC (18.92 grams) and oxyma (21.3 grams) were added to the resulting reaction mixture and stirred for 5-10 minutes at the same temperature. It was added to the resin obtained in step-A and stirred for 2-3 hours at room temperature. The progress of coupling was monitored by Kaiser test. After completion of reaction the resin was washed with DMF, isopropanol and dichloromethane.

Step-F: Fmoc-Ser(tBu)-OH (57.51 grams) was dissolved in DMF and stirred for 10 minutes. DIC (18.92 grams) and oxyma (21.3 grams) were added to the resulting reaction mixture and stirred for 5-10 minutes at the same temperature. It was added to the resin obtained in step-A and stirred for 2-3 hours at room temperature. The progress of coupling was monitored by Kaiser test. After completion of reaction the resin was washed with DMF, isopropanol and dichloromethane.

Step-G: Selective cleavage of CTC-resin from Fmoc-Ser(tBu)-Tyr(tBu)-Leu-Glu(OtBu)-Gly-CTC resin was performed with a mixture of Trifluoroacetic acid in dichloromethane. The crude protected peptide was isolated by precipitating with ether.

Yield: 96.6%; Purity: 98.33%

Stage-4: Synthesis of Fmoc-Gln(Trt)-Ala-Ala-Lys(PEG-PEG-γ-Glu-octadecane dioic acid)-Glu(OtBu)-Phe-Ile-Ala-Trp(Boc)-OH [Fragment-4]

Step-A: CTC resin (50 grams) was taken in a SPPS reactor and dichloromethane was added and allowed it to swell for 10 minutes.

Step-B: A solution of Fmoc-Trp(Boc)-OH (16.84 grams) and Diisopropylethylamine (10.35 grams) in dry dichloromethane was added to the resin obtained from step-A and stirred for 2 hours at room temperature. The above resin was deblocked with 20% piperidine in DMF for 10-15 minutes and washed with DMF.

Step-C: Fmoc-Ala-OH (9.33 grams) was dissolved in DMF and stirred for 10 minutes. DIC (3.79 grams) and oxyma (4.26 grams) were added to the resulting reaction mixture and stirred for 5-10 minutes at the room temperature. It was added to the resin obtained in step-A and stirred for 2-3 hours at room temperature. The progress of coupling was monitored by Kaiser tests. After completion of the reaction, the resin was drained and washed with DMF, isopropanol and dichloromethane. The resulting resin was deblocked with 20% piperidine in DMF for 10 minutes and washed with DMF.

Step-D: Fmoc-Ile-OH (10.60 grams) was dissolved in DMF and stirred for 10 minutes. DIC (3.79 grams) and oxyma (4.26 grams) were added to the resulting reaction mixture and stirred for 5-10 minutes at the same temperature. It was added to the resin obtained in step-A and stirred for 2-3 hours at room temperature. The progress of coupling was monitored by Kaiser test. After completion of reaction the resin was drained and washed with DMF, isopropanol and dichloromethane. The resulting resin was deblocked with 20% piperidine in DMF.

Step-E: Fmoc-Phe-OH (11.61 grams) was dissolved in DMF and stirred for 10 minutes. DIC (3.79 grams) and oxyma (4.26 grams) were added to the resulting reaction mixture and stirred for 5-10 minutes at the same temperature. It was added to the resin obtained in step-A and stirred for 2-3 hours at room temperature. The progress of coupling was monitored by Kaiser test. After completion of reaction the resin was washed with DMF, isopropanol and dichloromethane.

Step-F: Fmoc-Glu(OtBu)-OH (12.76 grams) was dissolved in DMF and stirred for 10 minutes. DIC (3.79 grams) and oxyma (4.26 grams) were added to the resulting reaction mixture and stirred for 5-10 minutes at the same temperature. It was added to the resin obtained in step-A and stirred for 2-3 hours at room temperature. The progress of coupling was monitored by Kaiser test. After completion of reaction, the resin was washed with DMF, isopropanol and dichloromethane.

Step-G: Fmoc-Lys(C18 diacid mono-t-butyl-γ-Glu(AEEA-AEEA)-OtBu)-OH (38.6 grams) was dissolved in DMF and stirred for 10 minutes. DIC (3.79 grams) and oxyma (4.26 grams) were added to the resulting reaction mixture and stirred for 5-10 minutes at the same temperature. It was added to the resin obtained in step-A and stirred for 2-3 hours at room temperature. The progress of coupling was monitored by Kaiser test. After completion of reaction, the resin was drained and washed with DMF, isopropanol and dichloromethane. The resulting resin was deblocked with 20% piperidine in DMF.

Step-H: Fmoc-Ala-OH (9.33 grams) was dissolved in DMF and stirred for 10 minutes. DIC (3.79 grams) and oxyma (4.26 grams) were added to the resulting reaction mixture and stirred for 5-10 minutes at the same temperature. It was added to the resin obtained in step-A and stirred for 2-3 hours at room temperature. The progress of coupling was monitored by Kaiser test. After completion of reaction, the resin was washed with DMF, isopropanol and dichloromethane.

Step-I: Fmoc-Ala-OH (9.33 grams) was dissolved in DMF and stirred for 10 minutes. DIC (3.79 grams) and oxyma (4.26 grams) were added to the resulting reaction mixture and stirred for 5-10 minutes at the same temperature. It was added to the resin obtained in step-A and stirred for 2-3 hours at room temperature. The progress of coupling was monitored by Kaiser test. After completion of reaction, the resin was washed with DMF, isopropanol and dichloromethane.

Step-J: Fmoc-Gln(Trt)-OH (18.32 grams) was dissolved in DMF and stirred for 10 minutes. DIC (3.79 grams) and oxyma (4.26 grams) were added to the resulting reaction mixture and stirred for 5-10 minutes at the same temperature. It was added to the resin obtained in step-A and stirred for 2-3 hours at room temperature. The progress of coupling was monitored by Kaiser test. After completion of reaction, the resin was washed with DMF, isopropanol and dichloromethane.

Step-K: Selective cleavage of CTC-resin from Fmoc-Gln(Trt)-Ala-Ala-Lys(C18 diacid mono-t-butyl-γ-Glu(AEEA-AEEA)-OtBu)-Glu(OtBu)-Phe-Ile-Ala-Trp(Boc)-CTC resin was performed with a mixture of Trifluoroacetic acid in dichloromethane. The crude protected peptide was isolated by precipitating with ether.

Yield: 95.0%; Purity: 92.1%

Stage-5: Synthesis of H-Leu-Val-Arg(Pbf)-Gly-Arg(Pbf)-Gly-OtBu [Fragment-5]

Step-A: CTC resin (50 grams) was taken in a SPPS reactor and dichloromethane was added and allowed it to swell for 10 minutes.

Step-B: A solution of Fmoc-Arg(Pbf)-OH (129.76 grams) and Diisopropylethylamine (69.85 grams) in dry dichloromethane was added to the resin obtained from step-A and stirred for 2 hours at room temperature. The above resin was deblocked with 20% piperidine in DMF for 10-15 minutes and washed with DMF.

Step-C: Fmoc-Gly-OH (44.59 grams) was dissolved in DMF and stirred for 10 minutes. DIC (23.2 grams) and oxyma (21.3 grams) were added to the resulting reaction mixture and stirred for 5-10 minutes at the room temperature. It was added to the resin obtained in step-A and stirred for 2-3 hours at room temperature. The progress of coupling was monitored by Kaiser tests. After completion of the reaction, the resin was drained and washed with DMF, isopropanol and dichloromethane. The resulting resin was deblocked with 20% piperidine in DMF for 10 minutes and washed with DMF.

Step-D: Fmoc-Arg(Pbf)-OH (97.3 grams) was dissolved in DMF and stirred for 10 minutes. DIC (23.2 grams) and oxyma (21.3 grams) were added to the resulting reaction mixture and stirred for 5-10 minutes at the same temperature. It was added to the resin obtained in step-A and stirred for 2-3 hours at room temperature. The progress of coupling was monitored by Kaiser test. After completion of reaction, the resin was drained and washed with DMF, isopropanol and dichloromethane. The resulting resin was deblocked with 20% piperidine in DMF.

Step-E: Fmoc-Val-OH (50.9 grams) was dissolved in DMF and stirred for 10 minutes. DIC (23.2 grams) and oxyma (21.3 grams) were added to the resulting reaction mixture and stirred for 5-10 minutes at the same temperature. It was added to the resin obtained in step-A and stirred for 2-3 hours at room temperature. The progress of coupling was monitored by Kaiser test. After completion of reaction, the resin was washed with DMF, isopropanol and dichloromethane.

Step-F: Fmoc-Leu-OH (53 grams) was dissolved in DMF and stirred for 10 minutes. DIC (23.2 grams) and oxyma (21.3 grams) were added to the resulting reaction mixture and stirred for 5-10 minutes at the same temperature. It was added to the resin obtained in step-A and stirred for 2-3 hours at room temperature. The progress of coupling was monitored by Kaiser test. After completion of reaction, the resin was washed with DMF, isopropanol and dichloromethane.

Step-G: Selective cleavage of CTC-resin from Fmoc-Leu-Val-Arg(Pbf)-Gly-Arg(Pbf)-CTC resin was performed with a mixture of Trifluoroacetic acid and TIPS in dichloromethane. The crude protected peptide was isolated by precipitating with ether.

Step-H: Resin and peptide obtained from step-G were taken in a SPPS reactor and N,N-Dimethyl formamide was added and allowed it to swell for 10 minutes. Gly-OtBu. HCl (6.57 grams) is added in presence of EDC·HCl (7.59 grams) and NMM (3.48 grams) at 25-30° C. and stirred for 2-3 hours at the same temperature. Cooled the resulting reaction mixture and water was added to it. Filtered the precipitated solid and washed with water.

Step-I: Selective cleavage of resin from protected peptide resin obtained from step-H was performed with tert-butyl amine (35.1 grams) in n-heptane. The crude peptide was extracted with ethyl acetate and washed with water followed by brine solution. Filtered the precipitated peptide.

Yield: 74%; Purity: 96%

### Example-2: Process for the Preparation of Semaglutide by Using Solution Phase Peptide Synthesis Approach

Step-A: Fmoc-Gln(Trt)-Ala-Ala-Lys(C18 diacid mono-t-butyl-γ-Glu(AEEA-AEEA)-OtBu)-Glu(OtBu)-Phe-Ile-Ala-Trp(Boc)-OH (Fragment-4) was dissolved in DMF and stirred for 10 minutes at 25-30° C. H-Leu-Val-Arg(Pbf)-Gly-Arg(Pbf)-Gly-OtBu (Fragment-5), EDC·HCl and HOBT in DCM were added to the resulting reaction mixture at 25-30° C. and stirred for 15-20 minutes at the same temperature. Precipitated solid was filtered and washed with water and hexane. The resulting protected peptide was deprotected with tert-butyl amine and n-heptane in DMF. Filtered the precipitated solid and washed with water, hexane and methanol to get Gln(Trt)-Ala-Ala-Lys(C18 diacid mono-t-butyl-γ-Glu(AEEA-AEEA)-OtBu)-Glu(OtBu)-Phe-Ile-Ala-Trp (Boc)-Leu-Val-Arg (Pbf)-Gly-Arg(Pbf)-Gly-OtBu.

Step-B: Fmoc-Ser(tBu)-Tyr(tBu)-Leu-Glu(OtBu)-Gly-OH (Fragment-3) was dissolved in DMF and stirred for 10 minutes. H-Protected 16 amino acid peptide obtained in step-A was added in presence of EDC·HCl and HOBT in DCM at 25-30° C. and stirred for 15-20 minutes at the same temperature. Precipitated solid was filtered and washed with water and hexane followed by dried under vacuum for 2 hours. The resulting protected peptide was deprotected with tert-butyl amine and n-heptane in DMF. Filtered the precipitated solid and washed with water, hexane and methanol to get Ser(tBu)-Tyr(tBu)-Leu-Glu(OtBu)-Gly-Gln(Trt)-Ala-Ala-Lys(C18 diacid mono-t-butyl-γ-Glu(AEEA-AEEA)-OtBu)-Glu(OtBu)-Phe-Ile-Ala-Trp(Boc)-Leu-Val-Arg(Pbf)-Gly-Arg(Pbf)-Gly-OtBu.

Step-C: Fmoc-Thr(tBu)-Phe-Thr(tBu)-Ser(tBu)-Asp(OtBu)-Val-Ser(Oxa)-OH (Fragment-2) was dissolved in DMF and stirred for 10 minutes. Peptide obtained in step-B was added in presence of EDC·HCl and HOBT in DCM at 25-30° C. and stirred for 15-20 minutes at the same temperature. Precipitated solid was filtered and washed with water and hexane followed by dried under vacuum for 2 hours. The resulting protected peptide was deprotected with tert-butyl amine and n-heptane in DMF. Filtered the precipitated solid and washed with water, hexane and methanol to get Thr(tBu)-Phe-Thr(tBu)-Ser(tBu)-Asp(OtBu)-Val-Ser(Oxa)-Ser(tBu)-Tyr(tBu)-Leu-Glu(OtBu)-Gly-Gln(Trt)-Ala-Ala-Lys(C18 diacid mono-t-butyl-γ-Glu(AEEA-AEEA)-OtBu)-Glu(OtBu)-Phe-Ile-Ala-Trp(Boc)-Leu-Val-Arg(Pbf)-Gly-Arg(Pbf)-Gly-OtBu.

Step-D: Boc-His(Trt)-Aib-Glu(OtBu)-Gly-OH (Fragment-1) was dissolved in DMF and stirred for 10 minutes. Peptide obtained in step-C was added in presence of EDC·HCl and HOBT in DCM at 25-30° C. and stirred for 15-20 minutes at the same temperature. Precipitated solid was filtered and washed with water and hexane followed by dried under vacuum for 2 hours to get Boc-protected peptide.

The resulting protected peptide was cleaved with a cocktail mixture of TFA, TIPS, water and DTT in presence of DCM at 10-15° C. and stirred for 3-6 hours at the same temperature. Chilled DIPE was added to the resulting mixture and stirred for 2 hours. The precipitated solid was filtered and washed with DCM followed by DIPE to get crude Semaglutide.

### Example-3

Preparation-1: Process for the Preparation of Boc-His(Trt)-Aib-Glu(OtBu)-Gly-Thr(tBu)-Phe-Thr(tBu)-Ser(tBu)-Asp(OtBu)-Val-Ser(Oxa)-OH (Fragment-7)

Step-A: CTC resin (20 grams) was taken in a SPPS reactor and dichloromethane (120 mL) was added and allowed it to swell for 10 minutes.

Step-B: A solution of Fmoc-Val-Ser(Oxa)-OH (41 grams) and Diisopropylethylamine (20.77 grams) in dry dichloromethane was added to the resin obtained from step-A and stirred for 2 hours at room temperature. The above resin was deblocked with 20% piperidine in DMF for 10-15 minutes and washed with DMF.

Step-C: Fmoc-Ser(tBu)-OH (18.49 grams) was dissolved in DMF and stirred for 10 minutes. DIC (6 grams) and oxyma (6.82 grams) were added to the resulting reaction mixture and stirred for 5-10 minutes at the room temperature. It was added to the resin obtained in step-A and stirred for 2-3 hours at room temperature. The progress of coupling was monitored by Kaiser tests. After completion of the reaction, the resin was drained and washed with DMF, isopropanol and dichloromethane. The resulting resin was deblocked with 20% piperidine in DMF for 10 minutes and washed with DMF.

Step-D: Fmoc-Thr(tBu)-OH (19.08 grams) was dissolved in DMF and stirred for 10 minutes. DIC (6 grams) and oxyma (6.82 grams) were added to the resulting reaction mixture and stirred for 5-10 minutes at the same temperature. It was added to the resin obtained in step-A and stirred for 2-3 hours at room temperature. The progress of coupling was monitored by Kaiser test. After completion of reaction the resin was drained and washed with DMF, isopropanol and dichloromethane. The resulting resin was deblocked with 20% piperidine in DMF.

Step-E: Fmoc-Phe-OH (18.60 grams) was dissolved in DMF and stirred for 10 minutes. DIC (6.0 grams) and oxyma (6.82 grams) were added to the resulting reaction mixture and stirred for 5-10 minutes at the same temperature. It was added to the resin obtained in step-A and stirred for 2-3 hours at room temperature. The progress of coupling was monitored by Kaiser test. After completion of reaction the resin was washed with DMF, isopropanol and dichloromethane. The resulting resin was deblocked with 20% piperidine in DMF.

Step-F: Fmoc-Thr(tBu)-OH (19.08 grams) was dissolved in DMF and stirred for 10 minutes. DIC (6.0 grams) and oxyma (6.82 grams) were added to the resulting reaction mixture and stirred for 5-10 minutes at the same temperature. It was added to the resin obtained in step-A and stirred for 2-3 hours at room temperature. The progress of coupling was monitored by Kaiser test. After completion of reaction the resin was washed with DMF, isopropanol and dichloromethane. The resulting resin was deblocked with 20% piperidine in DMF.

Step-G: Fmoc-Gly-OH (47.57 grams) was dissolved in DMF and stirred for 10 minutes. DIC (6.0 grams) and oxyma (6.82 grams) were added to the resulting reaction mixture and stirred for 5-10 minutes at the same temperature. It was added to the resin obtained in step-A and stirred for 2-3 hours at room temperature. The progress of coupling was monitored by Kaiser test. After completion of reaction the resin was washed with DMF, isopropanol and dichloromethane. The resulting resin was deblocked with 20% piperidine in DMF.

Step-H: Fmoc-Glu(OtBu)-OH (76.5 grams) was dissolved in DMF and stirred for 10 minutes. DIC (22.72 grams) and oxyma (25.56 grams) were added to the resulting reaction mixture and stirred for 5-10 minutes at the room temperature. It was added to the resin obtained in step-A and stirred for 2-3 hours at room temperature. The progress of coupling was monitored by Kaiser tests. After completion of the reaction, the resin was drained and washed with DMF, isopropanol and dichloromethane. The resulting resin was deblocked with 20% piperidine in DMF for 10 minutes and washed with DMF.

Step-I: Fmoc-Aib-OH (56.03 grams) was dissolved in DMF and stirred for 10 minutes. DIC (22.72 grams) and oxyma (25.56 grams) were added to the resulting reaction mixture and stirred for 5-10 minutes at the same temperature. It was added to the resin obtained in step-A and stirred for 2-3 hours at room temperature. The progress of coupling was monitored by Kaiser test. After completion of reaction the resin was drained and washed with DMF, isopropanol and dichloromethane. The resulting resin was deblocked with 20% piperidine in DMF.

Step-J: Boc-His(Trt)-OH (89.46 grams) was dissolved in DMF and stirred for 10 minutes. DIC (22.72 grams) and oxyma (25.56 grams) were added to the resulting reaction mixture and stirred for 5-10 minutes at the same temperature. It was added to the resin obtained in step-A and stirred for 2-3 hours at room temperature. The progress of coupling was monitored by Kaiser test. After completion of reaction the resin was washed with DMF, isopropanol and dichloromethane.

Step-K: Selective cleavage of CTC-resin from Boc-His(Trt)-Ala-Glu(OtBu)-Gly-Thr(tBu)-Phe-Thr(tBu)-Ser(tBu)-Asp(OtBu)-Val-Ser(Oxa)-CTC resin was performed with a mixture of 1% Trifluoroacetic acid in dichloromethane. The crude protected peptide was isolated by precipitating with ether.

Preparation-2: Alternative Process for the Preparation of Boc-His(Boc)-Aib-Glu(OtBu)-Gly-Thr(tBu)-Phe-Thr(tBu)-Ser(tBu)-Asp(OtBu)-Val-Ser(Oxa)-OH (Fragment-7)

Step-A: CTC resin (20 grams) was taken in a SPPS reactor and dichloromethane (120 mL) was added and allowed it to swell for 10 minutes.

Step-B: A solution of Fmoc-Val-Ser(Oxa)-OH (41 grams) and Diisopropylethylamine (20.77 grams) in dry dichloromethane was added to the resin obtained from step-A and stirred for 2 hours at room temperature. The above resin was deblocked with 20% piperidine in DMF for 10-15 minutes and washed with DMF.

Step-C: Fmoc-Ser(tBu)-OH (18.49 grams) was dissolved in DMF and stirred for 10 minutes. DIC (6 grams) and oxyma (6.82 grams) were added to the resulting reaction mixture and stirred for 5-10 minutes at the room temperature. It was added to the resin obtained in step-A and stirred for 2-3 hours at room temperature. The progress of coupling was monitored by Kaiser tests. After completion of the reaction, the resin was drained and washed with DMF, isopropanol and dichloromethane. The resulting resin was deblocked with 20% piperidine in DMF for 10 minutes and washed with DMF.

Step-D: Fmoc-Thr(tBu)-OH (19.08 grams) was dissolved in DMF and stirred for 10 minutes. DIC (6 grams) and oxyma (6.82 grams) were added to the resulting reaction mixture and stirred for 5-10 minutes at the same temperature. It was added to the resin obtained in step-A and stirred for 2-3 hours at room temperature. The progress of coupling was monitored by Kaiser test. After completion of reaction the resin was drained and washed with DMF, isopropanol and dichloromethane. The resulting resin was deblocked with 20% piperidine in DMF.

Step-E: Fmoc-Phe-OH (18.60 grams) was dissolved in DMF and stirred for 10 minutes. DIC (6.0 grams) and oxyma (6.82 grams) were added to the resulting reaction mixture and stirred for 5-10 minutes at the same temperature. It was added to the resin obtained in step-A and stirred for 2-3 hours at room temperature. The progress of coupling was monitored by Kaiser test. After completion of reaction the resin was washed with DMF, isopropanol and dichloromethane. The resulting resin was deblocked with 20% piperidine in DMF.

Step-F: Fmoc-Thr(tBu)-OH (19.08 grams) was dissolved in DMF and stirred for 10 minutes. DIC (6.0 grams) and oxyma (6.82 grams) were added to the resulting reaction mixture and stirred for 5-10 minutes at the same temperature. It was added to the resin obtained in step-A and stirred for 2-3 hours at room temperature. The progress of coupling was monitored by Kaiser test. After completion of reaction the resin was washed with DMF, isopropanol and dichloromethane. The resulting resin was deblocked with 20% piperidine in DMF.

Step-G: Fmoc-Gly-OH (47.57 grams) was dissolved in DMF and stirred for 10 minutes. DIC (6.0 grams) and oxyma (6.82 grams) were added to the resulting reaction mixture and stirred for 5-10 minutes at the same temperature. It was added to the resin obtained in step-A and stirred for 2-3 hours at room temperature. The progress of coupling was monitored by Kaiser test. After completion of reaction the resin was washed with DMF, isopropanol and dichloromethane. The resulting resin was deblocked with 20% piperidine in DMF.

Step-H: Fmoc-Glu(OtBu)-OH (76.5 grams) was dissolved in DMF and stirred for 10 minutes. DIC (22.72 grams) and oxyma (25.56 grams) were added to the resulting reaction mixture and stirred for 5-10 minutes at the room temperature. It was added to the resin obtained in step-A and stirred for 2-3 hours at room temperature. The progress of coupling was monitored by Kaiser tests. After completion of the reaction, the resin was drained and washed with DMF, isopropanol and dichloromethane. The resulting resin was deblocked with 20% piperidine in DMF for 10 minutes and washed with DMF.

Step-I: Fmoc-Aib-OH (56.03 grams) was dissolved in DMF and stirred for 10 minutes. DIC (22.72 grams) and oxyma (25.56 grams) were added to the resulting reaction mixture and stirred for 5-10 minutes at the same temperature. It was added to the resin obtained in step-A and stirred for 2-3 hours at room temperature. The progress of coupling was monitored by Kaiser test. After completion of reaction the resin was drained and washed with DMF, isopropanol and dichloromethane. The resulting resin was deblocked with 20% piperidine in DMF.

Step-J: Boc-His(Boc)-OH (89.46 grams) was dissolved in DMF and stirred for 10 minutes. DIC (22.72 grams) and oxyma (25.56 grams) were added to the resulting reaction mixture and stirred for 5-10 minutes at the same temperature. It was added to the resin obtained in step-A and stirred for 2-3 hours at room temperature. The progress of coupling was monitored by Kaiser test. After completion of reaction the resin was washed with DMF, isopropanol and dichloromethane.

Step-K: Selective cleavage of CTC-resin from Boc-His(Boc)-Ala-Glu(OtBu)-Gly-Thr(tBu)-Phe-Thr(tBu)-Ser(tBu)-Asp(OtBu)-Val-Ser(Oxa)-CTC resin was performed with a mixture of 1% Trifluoroacetic acid in dichloromethane. The crude protected peptide was isolated by precipitating with ether.

### Example-4: Process for the Preparation of Semaglutide by Using Hybrid Approach [Three Fragment Approach]

Stage-1: Solid Phase Peptide Synthesis of Fmoc-Ser(tBu)-Tyr(tBu)-Leu-Glu(OtBu)-Gly-OH (Fragment-3)

Step-A: CTC resin (50 grams) was taken in a SPPS reactor and dichloromethane was added and allowed it to swell for 10 minutes.

Step-B: A solution of Fmoc-Gly-OH (71.4 grams) and Diisopropylethylamine (51.72 grams) in dry dichloromethane was added to the resin obtained from step-A and stirred for 2 hours at room temperature. The above resin was deblocked with 20% piperidine in DMF for 10-15 minutes and washed with DMF.

Step-C: Fmoc-Glu(OtBu)-OH (63.82 grams) was dissolved in DMF and stirred for 10 minutes. DIC (18.92 grams) and oxyma (21.3 grams) were added to the resulting reaction mixture and stirred for 5-10 minutes at the room temperature. It was added to the resin obtained in step-A and stirred for 2-3 hours at room temperature. The progress of coupling was monitored by Kaiser tests. After completion of the reaction, the resin was drained and washed with DMF, isopropanol and dichloromethane. The resulting resin was deblocked with 20% piperidine in DMF for 10 minutes and washed with DMF.

Step-D: Fmoc-Leu-OH (53.01 grams) was dissolved in DMF and stirred for 10 minutes. DIC (18.92 grams) and oxyma (21.30 grams) were added to the resulting reaction mixture and stirred for 5-10 minutes at the same temperature. It was added to the resin obtained in step-A and stirred for 2-3 hours at room temperature. The progress of coupling was monitored by Kaiser test. After completion of reaction the resin was drained and washed with DMF, isopropanol and dichloromethane. The resulting resin was deblocked with 20% piperidine in DMF.

Step-E: Fmoc-Tyr(tBu)-OH (69 grams) was dissolved in DMF and stirred for 10 minutes. DIC (18.92 grams) and oxyma (21.3 grams) were added to the resulting reaction mixture and stirred for 5-10 minutes at the same temperature. It was added to the resin obtained in step-A and stirred for 2-3 hours at room temperature. The progress of coupling was monitored by Kaiser test. After completion of reaction the resin was washed with DMF, isopropanol and dichloromethane.

Step-F: Fmoc-Ser(tBu)-OH (57.51 grams) was dissolved in DMF and stirred for 10 minutes. DIC (18.92 grams) and oxyma (21.3 grams) were added to the resulting reaction mixture and stirred for 5-10 minutes at the same temperature. It was added to the resin obtained in step-A and stirred for 2-3 hours at room temperature. The progress of coupling was monitored by Kaiser test. After completion of reaction the resin was washed with DMF, isopropanol and dichloromethane.

Step-G: Selective cleavage of CTC-resin from Fmoc-Ser(tBu)-Tyr(tBu)-Leu-Glu(OtBu)-Gly-CTC resin was performed with a mixture of Trifluoroacetic acid in dichloromethane. The crude protected peptide was isolated by precipitating with ether.

Stage-2: Solid Phase Peptide Synthesis of Gln(Trt)-Ala-Ala-Lys(C18diacidmono-t-butyl-γ-Glu(AEEA-AEEA)-OtBu)-Glu(OtBu)-Phe-Ile-Ala-Trp(Boc)-Leu-Val-Arg(pbf)-Gly-Arg (pbf)-Gly-OtBu (Fragment-6)

Step-A: CTC resin (50 grams) was taken in a SPPS reactor and dichloromethane was added and allowed it to swell for 10 minutes.

Step-B: A solution of Fmoc-Arg(Pbf)-OH (129.76 grams) and Diisopropylethylamine (69.85 grams) in dry dichloromethane was added to the resin obtained from step-A and stirred for 2 hours at room temperature. The above resin was deblocked with 20% piperidine in DMF for 10-15 minutes and washed with DMF.

Step-C: Fmoc-Gly-OH (44.59 grams) was dissolved in DMF and stirred for 10 minutes. DIC (23.2 grams) and oxyma (21.3 grams) were added to the resulting reaction mixture and stirred for 5-10 minutes at the room temperature. It was added to the resin obtained in step-A and stirred for 2-3 hours at room temperature. The progress of coupling was monitored by Kaiser tests. After completion of the reaction, the resin was drained and washed with DMF, isopropanol and dichloromethane. The resulting resin was deblocked with 20% piperidine in DMF for 10 minutes and washed with DMF.

Step-D: Fmoc-Arg(Pbf)-OH (97.3 grams) was dissolved in DMF and stirred for 10 minutes. DIC (23.2 grams) and oxyma (21.3 grams) were added to the resulting reaction mixture and stirred for 5-10 minutes at the same temperature. It was added to the resin obtained in step-A and stirred for 2-3 hours at room temperature. The progress of coupling was monitored by Kaiser test. After completion of reaction, the resin was drained and washed with DMF, isopropanol and dichloromethane. The resulting resin was deblocked with 20% piperidine in DMF.

Step-E: Fmoc-Val-OH (50.9 grams) was dissolved in DMF and stirred for 10 minutes. DIC (23.2 grams) and oxyma (21.3 grams) were added to the resulting reaction mixture and stirred for 5-10 minutes at the same temperature. It was added to the resin obtained in step-A and stirred for 2-3 hours at room temperature. The progress of coupling was monitored by Kaiser test. After completion of reaction, the resin was washed with DMF, isopropanol and dichloromethane. The resulting resin was deblocked with 20% piperidine in DMF.

Step-F: Fmoc-Leu-OH (53 grams) was dissolved in DMF and stirred for 10 minutes. DIC (23.2 grams) and oxyma (21.3 grams) were added to the resulting reaction mixture and stirred for 5-10 minutes at the same temperature. It was added to the resin obtained in step-A and stirred for 2-3 hours at room temperature. The progress of coupling was monitored by Kaiser test. After completion of reaction, the resin was washed with DMF, isopropanol and dichloromethane. The resulting resin was deblocked with 20% piperidine in DMF.

Step-G: A solution of Fmoc-Trp(Boc)-OH (16.84 grams) and Diisopropylethylamine (10.35 grams) in dry dichloromethane was added to the resin obtained from step-A and stirred for 2 hours at room temperature. The above resin was deblocked with 20% piperidine in DMF for 10-15 minutes and washed with DMF.

Step-H: Fmoc-Ala-OH (9.33 grams) was dissolved in DMF and stirred for 10 minutes. DIC (3.79 grams) and oxyma (4.26 grams) were added to the resulting reaction mixture and stirred for 5-10 minutes at the room temperature. It was added to the resin obtained in step-A and stirred for 2-3 hours at room temperature. The progress of coupling was monitored by Kaiser tests. After completion of the reaction, the resin was drained and washed with DMF, isopropanol and dichloromethane. The resulting resin was deblocked with 20% piperidine in DMF for 10 minutes and washed with DMF.

Step-I: Fmoc-Ile-OH (10.60 grams) was dissolved in DMF and stirred for 10 minutes. DIC (3.79 grams) and oxyma (4.26 grams) were added to the resulting reaction mixture and stirred for 5-10 minutes at the same temperature. It was added to the resin obtained in step-A and stirred for 2-3 hours at room temperature. The progress of coupling was monitored by Kaiser test. After completion of reaction the resin was drained and washed with DMF, isopropanol and dichloromethane. The resulting resin was deblocked with 20% piperidine in DMF.

Step-J: Fmoc-Phe-OH (11.61 grams) was dissolved in DMF and stirred for 10 minutes. DIC (3.79 grams) and oxyma (4.26 grams) were added to the resulting reaction mixture and stirred for 5-10 minutes at the same temperature. It was added to the resin obtained in step-A and stirred for 2-3 hours at room temperature. The progress of coupling was monitored by Kaiser test. After completion of reaction the resin was washed with DMF, isopropanol and dichloromethane. The resulting resin was deblocked with 20% piperidine in DMF.

Step-K: Fmoc-Glu(OtBu)-OH (12.76 grams) was dissolved in DMF and stirred for 10 minutes. DIC (3.79 grams) and oxyma (4.26 grams) were added to the resulting reaction mixture and stirred for 5-10 minutes at the same temperature. It was added to the resin obtained in step-A and stirred for 2-3 hours at room temperature. The progress of coupling was monitored by Kaiser test. After completion of reaction, the resin was washed with DMF, isopropanol and dichloromethane. The resulting resin was deblocked with 20% piperidine in DMF.

Step-L: Fmoc-Lys(C18diacidmono-t-butyl-γ-Glu(AEEA-AEEA)-OtBu)-OH (11.88 grams) was dissolved in DMF and stirred for 10 minutes. DIC (3.79 grams) and oxyma (4.26 grams) were added to the resulting reaction mixture and stirred for 5-10 minutes at the same temperature. It was added to the resin obtained in step-A and stirred for 2-3 hours at room temperature. The progress of coupling was monitored by Kaiser test. After completion of reaction, the resin was drained and washed with DMF, isopropanol and dichloromethane. The resulting resin was deblocked with 20% piperidine in DMF. The resulting resin was deblocked with 20% piperidine in DMF.

Step-M: Fmoc-Ala-OH (9.33 grams) was dissolved in DMF and stirred for 10 minutes. DIC (3.79 grams) and oxyma (4.26 grams) were added to the resulting reaction mixture and stirred for 5-10 minutes at the same temperature. It was added to the resin obtained in step-A and stirred for 2-3 hours at room temperature. The progress of coupling was monitored by Kaiser test. After completion of reaction, the resin was washed with DMF, isopropanol and dichloromethane. The resulting resin was deblocked with 20% piperidine in DMF.

Step-N: Fmoc-Ala-OH (9.33 grams) was dissolved in DMF and stirred for 10 minutes. DIC (3.79 grams) and oxyma (4.26 grams) were added to the resulting reaction mixture and stirred for 5-10 minutes at the same temperature. It was added to the resin obtained in step-A and stirred for 2-3 hours at room temperature. The progress of coupling was monitored by Kaiser test. After completion of reaction, the resin was washed with DMF, isopropanol and dichloromethane. The resulting resin was deblocked with 20% piperidine in DMF.

Step-O: Fmoc-Gln(Trt)-OH (18.32 grams) was dissolved in DMF and stirred for 10 minutes. DIC (3.79 grams) and oxyma (4.26 grams) were added to the resulting reaction mixture and stirred for 5-10 minutes at the same temperature. It was added to the resin obtained in step-A and stirred for 2-3 hours at room temperature. The progress of coupling was monitored by Kaiser test. After completion of reaction, the resin was washed with DMF, isopropanol and dichloromethane.

Step-P: H-Gly-OtBu·HCl was dissolved in DMF and stirred for 10 minutes. EDC·HCl and HOAt were added to the resulting reaction mixture and stirred for 1-2 hours at the same temperature. It was added to the resin obtained in step-A and stirred for 1-2 hours. The obtain protected 15 Amino acid peptide chain was dissolved in DMF and cooled to 5-10° C. Tert-butyl amine was added to the resulting solution. Water was added to the resulting reaction mixture to obtain Fragment-6.

Stage-3: Synthesis of Semaglutide by Solution Phase Peptide Fragment Condensation:

Step-A: Gln(Trt)-Ala-Ala-Lys(C18diacid mono-t-butyl-γ-Glu(AEEA-AEEA)-OtBu)-Glu(OtBu)-Phe-Ile-Ala-Trp(Boc)-Leu-Val-Arg(Pbf)-Gly-Arg(Pbf)-Gly-OtBu (3.7 grams) (fragment-6) was dissolved in DMF and stirred for 10 minutes at 25-30° C. Fmoc-Ser(tBu)-Tyr(tBu)-Leu-Glu(OtBu)-Gly-OH (1.37 grams) (fragment-3), EDC·HCl and HOAT in DCM were added to the resulting reaction mixture at 25-30° C. and stirred for 15-20 minutes at the same temperature. Precipitated solid was filtered and washed with water and hexane. The resulting protected peptide was deprotected with tert-butylamine. Filtered the precipitated solid and washed with water and hexane to get Ser(tBu)-Tyr(tBu)-Leu-Glu(OtBu)-Gly-Gln(Trt)-Ala-Ala-Lys(C18diacidmono-t-butyl-γ-Glu(AEEA-AEEA)-OtBu)-Glu(OtBu)-Phe-Ile-Ala-Trp(Boc)-Leu-Val-Arg(Pbf)-Gly-Arg(Pbf)-Gly-OtBu.

Step-B: Boc-His(X)-Aib-Glu(OtBu)-Gly-Thr(tBu)-Phe-Thr(tBu)-Ser(tBu)-Asp(OtBu)-Val-Ser (Oxa)-OH (Fragment-7); wherein X is Boc or Trt (obtained from preparation-1 or -2) was dissolved in DMF and stirred for 10 minutes. H-Protected 20 amino acid peptide obtained in step-A was added in presence of EDC·HCl and HOAT in DCM at 25-30° C. and stirred for 1-2 hours at the same temperature. Precipitated solid was filtered and washed with water and hexane followed by dried under vacuum at 40-45° C. for 2 hours. The resulting protected peptide was cleaved with a cocktail mixture of TFA, TIPS, water and DTT in presence of DCM at 25-30° C. and stirred for 3-6 hours at the same temperature. Chilled DIPE was added to the resulting mixture and stirred for 2 hours. The precipitated solid was filtered and washed with DCM followed by DIPE to get crude Liraglutide.

Stage-C: Preparative HPLC Purification of Semaglutide

Crude Semaglutide obtained in step-B was dissolved in purified water and 25% aqueous ammonia and loaded on to preparative YMC Triart (50×250 mm, 10 μm). The peptide was purified using a linear gradient of trifluoro acetic acid and acetonitrile:water with flow rate of 50 mL/minute.

The pure fraction containing the Semaglutide was pooled.

It is diluted with purified water and purified using a linear gradient of water, ammonium bicarbonate and acetonitrile:water with a flow rate of 50 mL/minute. Volatiles were removed under reduced pressure and aqueous layer was lyophilized to give Semaglutide as a powder. The resulting peptide was analysed by RP-HPLC and confirmed by MALDI or LC-MS.

Purity: 97.20%

Preparation-3: Synthesis of Boc-His(Boc)-Aib-Glu(OtBu)-Gly-OH (Fragment-1)

Step-A: CTC resin (50 grams) was taken in a SPPS reactor and dichloromethane was added and allowed it to swell for 10 minutes.

Step-B: A solution of Fmoc-Gly-OH (71.3 grams) and Diisopropylethylamine (51.69 grams) in dry dichloromethane was added to the resin obtained from step-A and stirred for 2 hours at room temperature. The above resin was deblocked with 20% piperidine in DMF for 10-15 minutes and washed with DMF.

Step-C: Fmoc-Glu(OtBu)-OH (82.8 grams) was dissolved in DMF and stirred for 10 minutes. DIC (30.1 grams) and oxyma (27.69 grams) were added to the resulting reaction mixture and stirred for 5-10 minutes at the room temperature. It was added to the resin obtained in step-A and stirred for 2-3 hours at room temperature. The progress of coupling was monitored by Kaiser tests. After completion of the reaction, the resin was drained and washed with DMF, isopropanol and dichloromethane. The resulting resin was deblocked with 20% piperidine in DMF for 10 minutes and washed with DMF.

Step-D: Fmoc-Aib-OH (63.4 grams) was dissolved in DMF and stirred for 10 minutes. DIC (30.1 grams) and oxyma (27.69 grams) were added to the resulting reaction mixture and stirred for 5-10 minutes at the same temperature. It was added to the resin obtained in step-A and stirred for 2-3 hours at room temperature. The progress of coupling was monitored by Kaiser test. After completion of reaction the resin was drained and washed with DMF, isopropanol and dichloromethane. The resulting resin was deblocked with 20% piperidine in DMF.

Step-E: Boc-His(Boc)-OH (96.91 grams) was dissolved in DMF and stirred for 10 minutes. DIC (30.1 grams) and oxyma (27.69 grams) were added to the resulting reaction mixture and stirred for 5-10 minutes at the same temperature. It was added to the resin obtained in step-A and stirred for 2-3 hours at room temperature. The progress of coupling was monitored by Kaiser test. After completion of reaction the resin was washed with DMF, isopropanol and dichloromethane.

Step-F: Selective cleavage of CTC-resin from Boc-His(Boc)-Aib-Glu(OtBu)-Gly-CTC resin was performed with a mixture of 1% Trifluoroacetic acid in dichloromethane. The crude protected peptide was isolated by precipitating with ether.

Yield: 35 grams; Purity: 96%

### Example-2: Process for the Preparation of Semaglutide by Using Hybrid Approach [Four Fragment Approach]

Stage-1: Synthesis of Semaglutide by solution phase peptide fragment condensation:

Step-A: Gln(Trt)-Ala-Ala-Lys(C18diacidmono-t-butyl-γ-Glu(AEEA-AEEA)-OtBu)-Glu(OtBu)-Phe-Ile-Ala-Trp(Boc)-Leu-Val-Arg(Pbf)-Gly-Arg(Pbf)-Gly-OtBu (Fragment-6) obtained from stage-3 of example-1 was dissolved in DMF and stirred for 10 minutes at 25-30° C. Fmoc-Ser(tBu)-Tyr(tBu)-Leu-Glu(OtBu)-Gly-OH (Fragment-3) obtained from stage-2 of example-1, EDC·HCl and HOAT in DMF were added to the resulting reaction mixture at 25-30° C. and stirred for 15-20 minutes at the same temperature. Precipitated solid was filtered and washed with water and hexane. The resulting protected peptide was deprotected with tert-butylamine. Filtered the precipitated solid and washed with water and hexane to get Ser(tBu)-Tyr(tBu)-Leu-Glu(OtBu)-Gly-Gln(Trt)-Ala-Ala-Lys(C18diacidmono-t-butyl-γ-Glu(AEEA-AEEA)-OtBu)-Glu(OtBu)-Phe-Ile-Ala-Trp(Boc)-Leu-Val-Arg(Pbf)-Gly-Arg(Pbf)-Gly-OtBu.

Step-B: Fmoc-Thr(tBu)-Phe-Thr(tBu)-Ser(tBu)-Asp(OtBu)-Val-Ser(Oxa)-OH (Fragment-2) was dissolved in DMF and stirred for 10 minutes. H-Protected 20 amino acid peptide obtained in step-A was added in presence of EDC·HCl and HOAT in DMF at 25-30° C. and stirred for 15-20 minutes at the same temperature. Precipitated solid was filtered and washed with water and hexane. The resulting protected peptide was deprotected with tert-butylamine. Filtered the precipitated solid and washed with water and hexane to get Thr(tBu)-Phe-Thr(tBu)-Ser(tBu)-Asp(OtBu)-Val-Ser(Oxa)-Ser(tBu)-Tyr(tBu)-Leu-Glu(OtBu)-Gly-Gln(Trt)-Ala-Ala-Lys (C18 di acidmono-t-butyl-γ-Glu(AEEA-AEEA)-OtBu)-Glu(OtBu)-Phe-Ile-Ala-Trp(Boc)-Leu-Val-Arg (Pbf)-Gly-Arg(Pbf)-Gly-OtBu.

Step-C: Boc-His(X)-Aib-Glu(OtBu)-Gly-OH (Fragment-1); wherein X is Boc or Trt (obtained from preparation-1 or -2) was dissolved in DMF and stirred for 10 minutes. H-Protected amino acid peptide obtained in step-B was added in presence of EDC·HCl and HOAT in DMF at 25-30° C. and stirred for 15-20 minutes at the same temperature. Precipitated solid was filtered and washed with water and hexane followed by dried under vacuum for 2 hours. The resulting protected peptide was cleaved with a cocktail mixture of TFA, TIPS, water and DTT in presence of DCM at 10-15° C. and stirred for 3-6 hours at the same temperature. Chilled DIPE was added to the resulting mixture and stirred for 2 hours. The precipitated solid was filtered and washed with DCM followed by DIPE to get crude Semaglutide.

### Example-4: Alternative Solid Phase Peptide Synthesis of Gln(Trt)-Ala-Ala-Lys(C18diacid mono-t-butyl-γ-Glu(AEEA-AEEA)-OtBu)-Glu(OtBu)-Phe-Ile-Ala-Trp(Boc)-Leu-Val-Arg(pbf)-Gly-Arg (pbf)-Gly-OtBu (fragment-6)

Stage-1: Preparation of Fmoc-Gln(Trt)-Ala-Ala-Lys(C18diacid mono-t-butyl-γ-Glu (AEEA-AEEA)-OtBu)-OH

Step-A: CTC resin (50 grams) was taken in a SPPS reactor and dichloromethane was added and allowed it to swell for 10 minutes.

Step-B: A solution of Fmoc-Lys(C18diacid mono-t-butyl-γ-Glu (AEEA-AEEA)-OtBu)-OH and Diisopropylethylamine in dry dichloromethane was added to the resin obtained from step-A and stirred for 2 hours at room temperature. The above resin was deblocked with 20% piperidine in DMF for 10-15 minutes and washed with DMF.

Step-C: Fmoc-Ala-OH was dissolved in DMF and stirred for 10 minutes. DIC and oxyma were added to the resulting reaction mixture and stirred for 5-10 minutes at the room temperature. It was added to the resin obtained in step-A and stirred for 2-3 hours at room temperature. The progress of coupling was monitored by Kaiser tests. After completion of the reaction, the resin was drained and washed with DMF, isopropanol and dichloromethane. The resulting resin was deblocked with 20% piperidine in DMF for 10 minutes and washed with DMF.

Step-D: Fmoc-Ala-OH was dissolved in DMF and stirred for 10 minutes. DIC and oxyma were added to the resulting reaction mixture and stirred for 5-10 minutes at the room temperature. It was added to the resin obtained in step-A and stirred for 2-3 hours at room temperature. The progress of coupling was monitored by Kaiser tests. After completion of the reaction, the resin was drained and washed with DMF, isopropanol and dichloromethane. The resulting resin was deblocked with 20% piperidine in DMF for 10 minutes and washed with DMF.

Step-E: Fmoc-Gln(Trt)-OH was dissolved in DMF and stirred for 10 minutes. DIC and oxyma were added to the resulting reaction mixture and stirred for 5-10 minutes at the room temperature. It was added to the resin obtained in step-A and stirred for 2-3 hours at room temperature. The progress of coupling was monitored by Kaiser tests. After completion of the reaction, the resin was drained and washed with DMF, isopropanol and dichloromethane. The resulting resin was deblocked with 20% piperidine in DMF for 10 minutes and washed with DMF.

Step-F: Selective cleavage of CTC-resin from Fmoc-Gln(Trt)-Ala-Ala-Lys(C18diacid mono-t-butyl-γ-Glu (AEEA-AEEA)-OtBu)-CTC resin was performed with a mixture of 1% Trifluoro acetic acid in dichloromethane. The crude protected peptide was isolated by precipitating with ether.

Stage-2: Alternative Preparation of H-Gln (Trt)-Ala-Ala-Lys(C18diacidmono-t-butyl-γ-Glu(AEEA-AEEA)-OtBu)-Glu(OtBu)-Phe-Ile-Ala-Trp (Boc)-Leu-Val-Arg (Pbf)-Gly-Arg (Pbf)-Gly-OtBu

Step-A: CTC resin (50 grams) was taken in a SPPS reactor and dichloromethane was added and allowed it to swell for 10 minutes.

Step-B: A solution of Fmoc-Arg(Pbf)-OH (129.76 grams) and Diisopropylethylamine (69.85 grams) in dry dichloromethane was added to the resin obtained from step-A and stirred for 2 hours at room temperature. The above resin was deblocked with 20% piperidine in DMF for 10-15 minutes and washed with DMF.

Step-C: Fmoc-Gly-OH (44.59 grams) was dissolved in DMF and stirred for 10 minutes. DIC (23.2 grams) and oxyma (21.3 grams) were added to the resulting reaction mixture and stirred for 5-10 minutes at the room temperature. It was added to the resin obtained in step-A and stirred for 2-3 hours at room temperature. The progress of coupling was monitored by Kaiser tests. After completion of the reaction, the resin was drained and washed with DMF, isopropanol and dichloromethane. The resulting resin was deblocked with 20% piperidine in DMF for 10 minutes and washed with DMF.

Step-D: Fmoc-Arg(Pbf)-OH (97.3 grams) was dissolved in DMF and stirred for 10 minutes. DIC (23.2 grams) and oxyma (21.3 grams) were added to the resulting reaction mixture and stirred for 5-10 minutes at the same temperature. It was added to the resin obtained in step-A and stirred for 2-3 hours at room temperature. The progress of coupling was monitored by Kaiser test. After completion of reaction, the resin was drained and washed with DMF, isopropanol and dichloromethane. The resulting resin was deblocked with 20% piperidine in DMF.

Step-E: Fmoc-Val-OH (50.9 grams) was dissolved in DMF and stirred for 10 minutes. DIC (23.2 grams) and oxyma (21.3 grams) were added to the resulting reaction mixture and stirred for 5-10 minutes at the same temperature. It was added to the resin obtained in step-A and stirred for 2-3 hours at room temperature. The progress of coupling was monitored by Kaiser test. After completion of reaction, the resin was washed with DMF, isopropanol and dichloromethane. The resulting resin was deblocked with 20% piperidine in DMF.

Step-F: Fmoc-Leu-OH (53 grams) was dissolved in DMF and stirred for 10 minutes. DIC (23.2 grams) and oxyma (21.3 grams) were added to the resulting reaction mixture and stirred for 5-10 minutes at the same temperature. It was added to the resin obtained in step-A and stirred for 2-3 hours at room temperature. The progress of coupling was monitored by Kaiser test. After completion of reaction, the resin was washed with DMF, isopropanol and dichloromethane. The resulting resin was deblocked with 20% piperidine in DMF.

Step-G: A solution of Fmoc-Trp(Boc)-OH (16.84 grams) and Diisopropylethylamine (10.35 grams) in dry dichloromethane was added to the resin obtained from step-A and stirred for 2 hours at room temperature. The above resin was deblocked with 20% piperidine in DMF for 10-15 minutes and washed with DMF.

Step-H: Fmoc-Ala-OH (9.33 grams) was dissolved in DMF and stirred for 10 minutes. DIC (3.79 grams) and oxyma (4.26 grams) were added to the resulting reaction mixture and stirred for 5-10 minutes at the room temperature. It was added to the resin obtained in step-A and stirred for 2-3 hours at room temperature. The progress of coupling was monitored by Kaiser tests. After completion of the reaction, the resin was drained and washed with DMF, isopropanol and dichloromethane. The resulting resin was deblocked with 20% piperidine in DMF for 10 minutes and washed with DMF.

Step-I: Fmoc-Ile-OH (10.60 grams) was dissolved in DMF and stirred for 10 minutes. DIC (3.79 grams) and oxyma (4.26 grams) were added to the resulting reaction mixture and stirred for 5-10 minutes at the same temperature. It was added to the resin obtained in step-A and stirred for 2-3 hours at room temperature. The progress of coupling was monitored by Kaiser test. After completion of reaction the resin was drained and washed with DMF, isopropanol and dichloromethane. The resulting resin was deblocked with 20% piperidine in DMF.

Step-J: Fmoc-Phe-OH (11.61 grams) was dissolved in DMF and stirred for 10 minutes. DIC (3.79 grams) and oxyma (4.26 grams) were added to the resulting reaction mixture and stirred for 5-10 minutes at the same temperature. It was added to the resin obtained in step-A and stirred for 2-3 hours at room temperature. The progress of coupling was monitored by Kaiser test. After completion of reaction the resin was washed with DMF, isopropanol and dichloromethane. The resulting resin was deblocked with 20% piperidine in DMF.

Step-K: Fmoc-Glu(OtBu)-OH (12.76 grams) was dissolved in DMF and stirred for 10 minutes. DIC (3.79 grams) and oxyma (4.26 grams) were added to the resulting reaction mixture and stirred for 5-10 minutes at the same temperature. It was added to the resin obtained in step-A and stirred for 2-3 hours at room temperature. The progress of coupling was monitored by Kaiser test. After completion of reaction, the resin was washed with DMF, isopropanol and dichloromethane.

Step-L: H-Gly-OtBu·HCl was dissolved in DMF and stirred for 10 minutes. EDC·HCl and HOAt were added to the resulting reaction mixture and stirred for 1-2 hours at the same temperature. It was added to the resin obtained in step-A and stirred for 1-2 hours. The resulting resin was deblocked with 20% piperidine in DMF.

Fmoc-Gln(Trt)-Ala-Ala-Lys(C18diacid mono-t-butyl-γ-Glu(AEEA-AEEA)-OtBu)-OH obtained from stage-1 is added to the protected peptide obtained in step-L in presence of EDC·HCl and HOAt. The resulting reaction mixture was stirred for 1-2 hours at 25-30° C. Precipitated solid was filtered and washed with water and hexane. The resulting protected peptide was deprotected with tert-butylamine. Filtered the precipitated solid and washed with water and hexane to get H-Gln (Trt)-Ala-Ala-Lys(C18diacidmono-t-butyl-γ-Glu(AEEA-AEEA)-OtBu)-Glu(OtBu)-Phe-Ile-Ala-Trp (Boc)-Leu-Val-Arg (Pbf)-Gly-Arg (Pbf)-Gly-OtBu.

While the present disclosure has been illustrated and described with respect to a particular embodiment thereof, it should be appreciated by those of ordinary skill in the art that various modifications to this disclosure may be made without departing from the spirit and scope of the present disclosure.

1. A three fragment-based hybrid approach for the preparation of Semaglutide of formula-I.
His-Aib-Glu-Gly-Thr-Phe-Thr-Ser-Asp-Val-Ser-Ser-Tyr-Leu-Glu-Gly-Gln-Ala-Ala-Lys (C18 diacid mono-t-butyl-γ-Glu(AEEA-AEEA)-OtBu)-Glu-Phe-Ile-Ala-Trp-Leu-Val-Arg-Gly-Arg-Gly-OH Formula-I
which comprises:
a) synthesis of fragments-3, -6 and -7 on solid support;
Boc-His(X)-Aib-Glu(OtBu)-Gly-Thr(tBu)-Phe-Thr(tBu)-Ser(tBu)-Asp(OtBu)-Val-Ser (Oxa)-OH (fragment-7); wherein X is Boc or Trt
Fmoc-Ser(tBu)-Tyr(tBu)-Leu-Glu(OtBu)-Gly-OH (fragment-3);
H-Gln(Trt)-Ala-Ala-Lys(C18diacidmono-t-butyl-γ-Glu(AEEA-AEEA)-OtBu)-Glu (OtBu)-Phe-Ile-Ala-Trp(Boc)-Leu-Val-Arg(Pbf)-Gly-Arg(Pbf)-Gly-OtBu (fragment-6);
b) condensing H-Gln(Trt)-Ala-Ala-Lys(C18diacidmono-t-butyl-γ-Glu(AEEA-AEEA)-OtBu)-Glu(OtBu)-Phe-Ile-Ala-Trp(Boc)-Leu-Val-Arg(Pbf)-Gly-Arg(Pbf)-Gly-OtBu (fragment-6) with Fmoc-Ser(tBu)-Tyr(tBu)-Leu-Glu(OtBu)-Gly-OH (fragment-3) in presence of coupling agent and solvent in in-situ manner, followed by deprotection in presence of base to obtain H-Ser(tBu)-Tyr(tBu)-Leu-Glu(OtBu)-Gly-Gln(Trt)-Ala-Ala-Lys(C18diacidmono-t-butyl-γ-Glu(AEEA-AEEA)-OtBu)-Glu(OtBu)-Phe-Ile-Ala-Trp (Boc)-Leu-Val-Arg(pbf)-Gly-Arg (pbf)-Gly-OtBu;
c) condensing Boc-His(X)-Aib-Glu(OtBu)-Gly-Thr(tBu)-Phe-Thr(tBu)-Ser(tBu)-Asp (OtBu)-Val-Ser(Oxa)-OH (fragment-7) with peptide obtained in step-b) in presence of a coupling agent to obtain protected Semaglutide;
d) cleaving the protected Semaglutide using a reagent to obtain crude Semaglutide; and
e) purifying the crude Semaglutide by preparative HPLC to obtain pure Semaglutide.

2. A four fragment-based hybrid approach for the preparation of Semaglutide of formula-I.
His-Aib-Glu-Gly-Thr-Phe-Thr-Ser-Asp-Val-Ser-Ser-Tyr-Leu-Glu-Gly-Gln-Ala-Ala-Lys (C18 diacid mono-t-butyl-γ-Glu(AEEA-AEEA)-OtBu)-Glu-Phe-Ile-Ala-Trp-Leu-Val-Arg-Gly-Arg-Gly-OH Formula-I
which comprises:
a) synthesis of fragments-1, -2, -3 and -6 on solid support;
Boc-His(X)-Aib-Glu(OtBu)-Gly-OH (fragment-1); wherein X is Boc or Trt
Fmoc-Thr(tBu)-Phe-Thr(tBu)-Ser(tBu)-Asp(OtBu)-Val-Ser(Oxa)-OH (fragment-2);
Fmoc-Ser(tBu)-Tyr(tBu)-Leu-Glu(OtBu)-Gly-OH (fragment-3);
H-Gln(Trt)-Ala-Ala-Lys(C18diacidmono-t-butyl-γ-Glu(AEEA-AEEA)-OtBu)-Glu (OtBu)-Phe-Ile-Ala-Trp(Boc)-Leu-Val-Arg (Pbf)-Gly-Arg(Pbf)-Gly-OtBu (fragment-6);
b) condensing H-Gln(Trt)-Ala-Ala-Lys(C18diacidmono-t-butyl-γ-Glu(AEEA-AEEA)-OtBu)-Glu(OtBu)-Phe-Ile-Ala-Trp(Boc)-Leu-Val-Arg(Pbf)-Gly-Arg(Pbf)-Gly-OtBu (fragment-6) with Fmoc-Ser(tBu)-Tyr(tBu)-Leu-Glu(OtBu)-Gly-OH (fragment-3) in presence of coupling agent and solvent in in-situ manner, followed by deprotection in presence of base to obtain H-Ser(tBu)-Tyr(tBu)-Leu-Glu(OtBu)-Gly-Gln(Trt)-Ala-Ala-Lys(C18diacidmono-t-butyl-γ-Glu(AEEA-AEEA)-OtBu)-Glu(OtBu)-Phe-Ile-Ala-Trp (Boc)-Leu-Val-Arg(pbf)-Gly-Arg (pbf)-Gly-OtBu;
c) condensing Fmoc-Thr(tBu)-Phe-Thr(tBu)-Ser(tBu)-Asp(OtBu)-Val-Ser(Oxa)-OH (fragment-2) with peptide obtained in step-b) in presence of a coupling agent in in-situ manner, followed by deprotection in presence of base to obtain H-Thr(tBu)-Phe-Thr(tBu)-Ser(tBu)-Asp(OtBu)-Val-Ser(Oxa)-Ser(tBu)-Tyr(tBu)-Leu-Glu(OtBu)-Gly-Gln(Trt)-Ala-Ala-Lys(C18diacidmono-t-butyl-γ-Glu(AEEA-AEEA)-OtBu)-Glu(OtBu)-Phe-Ile-Ala-Trp(Boc)-Leu-Val-Arg(pbf)-Gly-Arg (pbf)-Gly-OtBu;
d) condensing Boc-His(X)-Aib-Glu(OtBu)-Gly-OH (fragment-1) with peptide obtained in step-c) in presence of a coupling agent in in-situ manner, followed by deprotection in presence of base to obtain protected Semaglutide;
e) cleaving the protected Semaglutide using a reagent to obtain crude Semaglutide; and
f) purifying the crude Semaglutide by preparative HPLC to obtain pure Semaglutide.

3. A five fragment-based hybrid approach for the preparation of Semaglutide of formula-I.
His-Aib-Glu-Gly-Thr-Phe-Thr-Ser-Asp-Val-Ser-Ser-Tyr-Leu-Glu-Gly-Gln-Ala-Ala-Lys (C18 diacid mono-t-butyl-γ-Glu(AEEA-AEEA)-OtBu)-Glu-Phe-Ile-Ala-Trp-Leu-Val-Arg-Gly-Arg-Gly-OH Formula-I
which comprises:
a) synthesis of fragments-1, -2, -3, -4 and -5 on solid support;
Boc-His(Trt)-Aib-Glu(OtBu)-Gly-OH (fragment-1);
Fmoc-Thr(tBu)-Phe-Thr(tBu)-Ser(tBu)-Asp(OtBu)-Val-Ser(Oxa)-OH (fragment-2);
Fmoc-Ser(tBu)-Tyr(tBu)-Leu-Glu(OtBu)-Gly-OH (fragment-3);
Fmoc-Gln(Trt)-Ala-Ala-Lys(C18 diacid mono-t-butyl-γ-Glu(AEEA-AEEA)-OtBu)-Glu (OtBu)-Phe-Ile-Ala-Trp(Boc)-OH (fragment-4);
Fmoc-Leu-Val-Arg(pbf)-Gly-Arg(pbf)-Gly-OtBu (fragment-5);
b) condensing Fmoc-Leu-Val-Arg(pbf)-Gly-Arg(pbf)-Gly-OtBu (fragment-5) with Fmoc-Gln(Trt)-Ala-Ala-Lys(PEG-PEG-7-Glu-octadecane dioic acid)-Glu (OtBu)-Phe-Ile-Ala-Trp(Boc)-OH (fragment-4) in presence of a coupling agent, followed by deprotection in presence of a base to obtain 15 amino acid peptide chain;
c) condensing Fmoc-Ser(tBu)-Tyr(tBu)-Leu-Glu(OtBu)-Gly-OH (fragment-3) with 15 amino acid peptide chain obtained in step-b) in presence of a coupling agent, followed by deprotection in presence of a base to obtain 20 amino acid peptide chain;
d) condensing Fmoc-Thr(tBu)-Phe-Thr(tBu)-Ser(tBu)-Asp(OtBu)-Val-Ser(Oxa)-OH (fragment-2) with 20 amino acid peptide chain obtained in step-c) in presence of a coupling agent, followed by deprotection in presence of a base to obtain 27 amino acid peptide chain;
e) condensing Boc-His(Trt)-Aib-Glu(OtBu)-Gly-OH (fragment-1) with 27 amino acid peptide chain obtained in stage-d) in presence of a coupling agent to obtain protected Semaglutide;
f) deprotecting the protected Semaglutide using a reagent to obtain crude Semaglutide; and
g) purifying the crude Semaglutide by preparative HPLC to obtain pure Semaglutide.

4. A solid phase peptide process for the preparation of Gln(Trt)-Ala-Ala-Lys(C18diacidmono-t-butyl-γ-Glu(AEEA-AEEA)-OtBu)-Glu(OtBu)-Phe-Ile-Ala-Trp(Boc)-Leu-Val-Arg(pbf)-Gly-Arg(pbf)-Gly-OtBu (fragment-6);
which comprises:
a) anchoring Fmoc-Arg(Pbf)-OH to a resin in presence of a base;
b) selective deprotection of amino acid using a base;
c) coupling of Fmoc-Gly-OH to a resin obtained in step-b) in presence of coupling agent in a solvent to obtain dipeptide resin;
d) sequential coupling of Fmoc-Arg(Pbf)-OH, Fmoc-Val-OH, Fmoc-Leu-OH, Fmoc-Trp(Boc)-OH, Fmoc-Ala-OH, Fmoc-Ile-OH, Fmoc-Phe-OH, Fmoc-Glu(OtBu)-OH, Fmoc-Lys(C18diacidmono-t-butyl-γ-Glu(AEEA-AEEA)-OtBu)-OH, Fmoc-Ala-OH, Fmoc-Ala-OH, Fmoc-Gln(Trt)-OH to the obtained resin in step-c) in presence of a coupling agent;
e) partial deprotection of peptide obtained in step-d) in presence of a reagent to obtain 14 amino acid chain peptide;
f) coupling of H-Gly-OtBu·HCl to 14 amino acid chain peptide obtained from step-e) in presence of coupling agent; and
g) deprotection of protected 15 amino acid peptide chain in step-f) in presence of reagent to obtain fragment-6.

5. A solid phase peptide process for the preparation of Gln(Trt)-Ala-Ala-Lys(C18diacidmono-t-butyl-γ-Glu(AEEA-AEEA)-OtBu)-Glu(OtBu)-Phe-Ile-Ala-Trp(Boc)-Leu-Val-Arg(pbf)-Gly-Arg(pbf)-Gly-OtBu (fragment-6)
which comprises:
a) anchoring Fmoc-Arg(Pbf)-OH to a resin in presence of a base;
b) selective deprotection of amino acid using a base;
c) coupling of Fmoc-Gly-OH to a resin obtained in step-b) in presence of coupling agent in a solvent to obtain dipeptide resin;
d) sequential coupling of Fmoc-Arg(Pbf)-OH, Fmoc-Val-OH, Fmoc-Leu-OH, Fmoc-Trp(Boc)-OH, Fmoc-Ala-OH, Fmoc-Ile-OH, Fmoc-Phe-OH, Fmoc-Glu(OtBu)-OH to the obtained resin in step-c) in presence of a coupling agent;
e) deprotection of protected peptide obtained in step-d) in presence of reagent to obtain Fmoc-Glu(OtBu)-Phe-Ile-Ala-Trp(Boc)-Leu-Val-Arg(Pbf)-Gly-Arg(Pbf)-OH;
f) coupling of H-Gly-OtBu·HCl to the peptide obtained in step-e) in presence of coupling agent to obtain 11 amino acid chain peptide;
g) coupling of Fmoc-Gln(Trt)-Ala-Ala-Lys(C18diacidmono-t-butyl-γ-Glu(AEEA-AEEA)-OtBu)-OH to the obtained 11 amino acid chain peptide in step-f) in presence of a coupling agent to obtain 15 amino acid chain peptide; and
h) partial deprotection of peptide obtained in step-g) in presence of a reagent to obtain fragment-6.

6. A solid phase peptide process for the preparation of Fmoc-Gln(Trt)-Ala-Ala-Lys(C18 diacid mono-t-butyl-γ-Glu(AEEA-AEEA)-OtBu)-Glu(OtBu)-Phe-Ile-Ala-Trp(Boc)-OH of fragment-4
which comprises:
a) anchoring Fmoc-Trp(Boc)-OH to a resin in presence of a coupling agent;
b) selective deprotection of amino acid using a base;
c) coupling of Fmoc-Ala-OH to a resin obtained in step-b) in presence of coupling agent in a solvent to obtain dipeptide resin;
d) sequential coupling of Fmoc-Ile-OH, Fmoc-Phe-OH, Fmoc-Glu(OtBu)-OH, Fmoc-Lys(PEG-PEG-γ-Glu-octadecane dioic acid)-OH, Fmoc-Ala-OH, Fmoc-Ala-OH, Fmoc-Gln(Trt)-OH to the obtained resin in step-a) in presence of a coupling agent; and
e) cleaving of protected peptide from solid support resin in presence of a reagent to get fragment-4.

7. The process as claimed in claim 1, wherein said coupling agent is selected from the group consisting of Ethylcyano (hydroxy imino) acetate-02)-tri-(1-pyrrol-idinyl)-Phosphonium hexa fluorophosphate (PyOxim), ethyl-2-cyano-2-(hydroxyamino) acetate (Oxyma pure), O-(benzotriazol-1-yl)-N,N,N′,N′-tetramethyluronium tetrafluoroborate (TBTU), diisopropyl carbodiimide (DIC), 1,3-dicyclohexylcabodiimide (DCC), O-(7-azabenzotriazol-1-yl)-N,N,N′,N′-tetramethyluronium hexafluorophosphate (HATU), 1-(dimethyl aminopropyl)-3-ethylcarbodiimide hydrochloride (EDC HCl), O-(benzotriazol-1-yl)-1,1,3,3-tetra methyluronium hexafluoro phosphate (HBTU), 1-Hydroxybenzotriazole (HOBt), 1-hydroxy-7-azabenzotriazole (HOAt), Isopropyl chloro formate (IPCF), Benzotriazol-1-yl-oxy-tris(dimethyl-amino)-phosphonium hexa fluorophosphate (BOP), benzotriazole-1-yloxy-tri(pyrrolidino) phosphonium hexa fluoro phosphate (PyBOP), N,N-bis-(2-oxo-3-oxazolidinyl)phosphonic dichloride (BOP-Cl), bromotri(pyrrolidino)phosphonium hexa fluoro phosphate (PyBrOP), O-(6-Chloro-1-hydrocibenzotriazol-1-yl)-1,1,3,3-tetramethyl uranium tetra fluoroborate (TCTU), chlorotri (pyrrolidino)phosphonium hexafluorophosphate (PyClOP), Ethyl 1,2-dihydro-2-ethoxyquinoline-carboxylate (EEDQ), isobutyl chloro formate (IBCF), 2-succinimido-1,1,3,3-tetramethyluronium tetrafluoro borate (TSTU), 1-Cyano-2-ethoxy-2-oxo ethylidene aminooxy) dimethyl amino morpholino-carbeniumhexafluorophosphate (COMU), 2-(5-norbornen-2,3-dicarboximido)-1,1,3,3-tetramethyluronium tetrafluoroborate (TNTU), propane phosphonic acid anhydride (PPAA), 3-(diethoxy phosphoryloxy)-1,2,3-benzotriazin-4(3H)-one (DEPBT) or its mixture.

8. The process as claimed claim 1, wherein said solvent used is selected from the group consisting of DMF, DCM, tetrahydrofuran, NMP, DMAC, methanol, ethanol, isopropanol, dichloroethane, 1,4-dioxane, ethyl acetate, acetonitrile, acetone or a mixture thereof.

9. The process as claimed in claim 1, wherein said base used for deprotection is selected from the group consisting of tert-butyl amine, 20% of 4-methyl piperidine in Dimethyl formamide, 20% of piperidine in Dimethyl formamide and 20% of piperazine in Dimethyl formamide.

10. The process as claimed in claim 1, wherein said reagent used in cleavage step is selected from the group consisting of TFA, TIPS, Water, DTT, Thioanisole, EDT, DMS, cresol, phenol, thiocresol, ammonium iodide, 2,2′-(ethylene dioxy)diethane or its mixture. Preferably using cocktail mixture of TFA, TIPS, water or DTT.

11. A novel fragment-4 used in the preparation of Semaglutide. Fmoc-Gln(Trt)-Ala-Ala-Lys(C18 diacid mono-t-butyl-γ-Glu(AEEA-AEEA)-OtBu)-Glu (OtBu)-Phe-Ile-Ala-Trp(Boc)-OH (fragment-4).

12. The process as claimed in claim 2, wherein said coupling agent is selected from the group consisting of Ethylcyano (hydroxy imino) acetate-02)-tri-(1-pyrrolidinyl)-Phosphonium hexa fluorophosphate (PyOxim), ethyl-2-cyano-2-(hydroxyamino) acetate (Oxyma pure), O-(benzotriazol-1-yl)-N,N,N′,N′-tetramethyluronium tetrafluoroborate (TBTU), diisopropyl carbodiimide (DIC), 1,3-dicyclohexylcabodiimide (DCC), O-(7-azabenzotriazol-1-yl)-N,N,N′,N′-tetramethyluronium hexafluorophosphate (HATU), 1-(dimethyl aminopropyl)-3-ethylcarbodiimide hydrochloride (EDC HCl), O-(benzotriazol-1-yl)-1,1,3,3-tetra methyluronium hexafluoro phosphate (HBTU), 1-Hydroxybenzotriazole (HOBt), 1-hydroxy-7-azabenzotriazole (HOAt), Isopropyl chloro formate (IPCF), Benzotriazol-1-yl-oxy-tris(dimethyl-amino)-phosphonium hexa fluorophosphate (BOP), benzotriazole-1-yloxytri(pyrrolidino) phosphonium hexa fluoro phosphate (PyBOP), N,N-bis-(2-oxo-3-oxazolidinyl)phosphonic dichloride (BOP-Cl), bromotri(pyrrolidino)phosphonium hexa fluoro phosphate (PyBrOP), O-(6-Chloro-1-hydrocibenzotriazol-1-yl)-1,1,3,3-tetramethyl uranium tetra fluoroborate (TCTU), chlorotri (pyrrolidino)phosphonium hexafluorophosphate (PyClOP), Ethyl 1,2-dihydro-2-ethoxyquinoline-carboxylate (EEDQ), isobutyl chloro formate (IBCF), 2-succinimido-1,1,3,3-tetramethyluronium tetrafluoro borate (TSTU), 1-Cyano-2-ethoxy-2-oxo ethylidene aminooxy) dimethyl amino morpholino-carbeniumhexafluorophosphate (COMU), 2-(5-norbornen-2,3-dicarboximido)-1,1,3,3-tetramethyluronium tetrafluoroborate (TNTU), propane phosphonic acid anhydride (PPAA), 3-(diethoxy phosphoryloxy)-1,2,3-benzotriazin-4(3H)-one (DEPBT) or its mixture.

13. The process as claimed in claim 3, wherein said coupling agent is selected from the group consisting of Ethylcyano (hydroxy imino) acetate-02)-tri-(1-pyrrolidinyl)-Phosphonium hexa fluorophosphate (PyOxim), ethyl-2-cyano-2-(hydroxyamino) acetate (Oxyma pure), O-(benzotriazol-1-yl)-N,N,N′,N′-tetramethyluronium tetrafluoroborate (TBTU), diisopropyl carbodiimide (DIC), 1,3-dicyclohexylcabodiimide (DCC), O-(7-azabenzotriazol-1-yl)-N,N,N′,N′-tetramethyluronium hexafluorophosphate (HATU), 1-(dimethyl aminopropyl)-3-ethylcarbodiimide hydrochloride (EDC HCl), O-(benzotriazol-1-yl)-1,1,3,3-tetra methyluronium hexafluoro phosphate (HBTU), 1-Hydroxybenzotriazole (HOBt), 1-hydroxy-7-azabenzotriazole (HOAt), Isopropyl chloro formate (IPCF), Benzotriazol-1-yl-oxy-tris(dimethyl-amino)-phosphonium hexa fluorophosphate (BOP), benzotriazole-1-yloxytri(pyrrolidino) phosphonium hexa fluoro phosphate (PyBOP), N,N-bis-(2-oxo-3-oxazolidinyl)phosphonic dichloride (BOP-Cl), bromotri(pyrrolidino)phosphonium hexa fluoro phosphate (PyBrOP), 0-(6-Chloro-1-hydrocibenzotriazol-1-yl)-1,1,3,3-tetramethyl uranium tetra fluoroborate (TCTU), chlorotri (pyrrolidino)phosphonium hexafluorophosphate (PyClOP), Ethyl 1,2-dihydro-2-ethoxyquinoline-carboxylate (EEDQ), isobutyl chloro formate (IBCF), 2-succinimido-1,1,3,3-tetramethyluronium tetrafluoro borate (TSTU), 1-Cyano-2-ethoxy-2-oxo ethylidene aminooxy) dimethyl amino morpholino-carbeniumhexafluorophosphate (COMU), 2-(5-norbornen-2,3-dicarboximido)-1,1,3,3-tetramethyluronium tetrafluoroborate (TNTU), propane phosphonic acid anhydride (PPAA), 3-(diethoxy phosphoryloxy)-1,2,3-benzotriazin-4(3H)-one (DEPBT) or its mixture.

14. The process as claimed in claim 4, wherein said coupling agent is selected from the group consisting of Ethylcyano (hydroxy imino) acetate-02)-tri-(1-pyrrolidinyl)-Phosphonium hexa fluorophosphate (PyOxim), ethyl-2-cyano-2-(hydroxyamino) acetate (Oxyma pure), O-(benzotriazol-1-yl)-N,N,N′,N′-tetramethyluronium tetrafluoroborate (TBTU), diisopropyl carbodiimide (DIC), 1,3-dicyclohexylcabodiimide (DCC), O-(7-azabenzotriazol-1-yl)-N,N,N′,N′-tetramethyluronium hexafluorophosphate (HATU), 1-(dimethyl aminopropyl)-3-ethylcarbodiimide hydrochloride (EDC HCl), O-(benzotriazol-1-yl)-1,1,3,3-tetra methyluronium hexafluoro phosphate (HBTU), 1-Hydroxybenzotriazole (HOBt), 1-hydroxy-7-azabenzotriazole (HOAt), Isopropyl chloro formate (IPCF), Benzotriazol-1-yl-oxy-tris(dimethyl-amino)-phosphonium hexa fluorophosphate (BOP), benzotriazole-1-yloxytri(pyrrolidino) phosphonium hexa fluoro phosphate (PyBOP), N,N-bis-(2-oxo-3-oxazolidinyl)phosphonic dichloride (BOP-Cl), bromotri(pyrrolidino)phosphonium hexa fluoro phosphate (PyBrOP), 0-(6-Chloro-1-hydrocibenzotriazol-1-yl)-1,1,3,3-tetramethyl uranium tetra fluoroborate (TCTU), chlorotri (pyrrolidino)phosphonium hexafluorophosphate (PyClOP), Ethyl 1,2-dihydro-2-ethoxyquinoline-carboxylate (EEDQ), isobutyl chloro formate (IBCF), 2-succinimido-1,1,3,3-tetramethyluronium tetrafluoro borate (TSTU), 1-Cyano-2-ethoxy-2-oxo ethylidene aminooxy) dimethyl amino morpholino-carbeniumhexafluorophosphate (COMU), 2-(5-norbornen-2,3-dicarboximido)-1,1,3,3-tetramethyluronium tetrafluoroborate (TNTU), propane phosphonic acid anhydride (PPAA), 3-(diethoxy phosphoryloxy)-1,2,3-benzotriazin-4(3H)-one (DEPBT) or its mixture.

15. The process as claimed in claim 5, wherein said coupling agent is selected from the group consisting of Ethylcyano (hydroxy imino) acetate-02)-tri-(1-pyrrolidinyl)-Phosphonium hexa fluorophosphate (PyOxim), ethyl-2-cyano-2-(hydroxyamino) acetate (Oxyma pure), O-(benzotriazol-1-yl)-N,N,N′,N′-tetramethyluronium tetrafluoroborate (TBTU), diisopropyl carbodiimide (DIC), 1,3-dicyclohexylcabodiimide (DCC), O-(7-azabenzotriazol-1-yl)-N,N,N′,N′-tetramethyluronium hexafluorophosphate (HATU), 1-(dimethyl aminopropyl)-3-ethylcarbodiimide hydrochloride (EDC HCl), O-(benzotriazol-1-yl)-1,1,3,3-tetra methyluronium hexafluoro phosphate (HBTU), 1-Hydroxybenzotriazole (HOBt), 1-hydroxy-7-azabenzotriazole (HOAt), Isopropyl chloro formate (IPCF), Benzotriazol-1-yl-oxy-tris(dimethyl-amino)-phosphonium hexa fluorophosphate (BOP), benzotriazole-1-yloxytri(pyrrolidino) phosphonium hexa fluoro phosphate (PyBOP), N,N-bis-(2-oxo-3-oxazolidinyl)phosphonic dichloride (BOP-Cl), bromotri(pyrrolidino)phosphonium hexa fluoro phosphate (PyBrOP), 0-(6-Chloro-1-hydrocibenzotriazol-1-yl)-1,1,3,3-tetramethyl uranium tetra fluoroborate (TCTU), chlorotri (pyrrolidino)phosphonium hexafluorophosphate (PyClOP), Ethyl 1,2-dihydro-2-ethoxyquinoline-carboxylate (EEDQ), isobutyl chloro formate (IBCF), 2-succinimido-1,1,3,3-tetramethyluronium tetrafluoro borate (TSTU), 1-Cyano-2-ethoxy-2-oxo ethylidene aminooxy) dimethyl amino morpholino-carbeniumhexafluorophosphate (COMU), 2-(5-norbornen-2,3-dicarboximido)-1,1,3,3-tetramethyluronium tetrafluoroborate (TNTU), propane phosphonic acid anhydride (PPAA), 3-(diethoxy phosphoryloxy)-1,2,3-benzotriazin-4(3H)-one (DEPBT) or its mixture.

16. The process as claimed in claim 6, wherein said coupling agent is selected from the group consisting of Ethylcyano (hydroxy imino) acetate-02)-tri-(1-pyrrolidinyl)-Phosphonium hexa fluorophosphate (PyOxim), ethyl-2-cyano-2-(hydroxyamino) acetate (Oxyma pure), O-(benzotriazol-1-yl)-N,N,N′,N′-tetramethyluronium tetrafluoroborate (TBTU), diisopropyl carbodiimide (DIC), 1,3-dicyclohexylcabodiimide (DCC), O-(7-azabenzotriazol-1-yl)-N,N,N′,N′-tetramethyluronium hexafluorophosphate (HATU), 1-(dimethyl aminopropyl)-3-ethylcarbodiimide hydrochloride (EDC HCl), O-(benzotriazol-1-yl)-1,1,3,3-tetra methyluronium hexafluoro phosphate (HBTU), 1-Hydroxybenzotriazole (HOBt), 1-hydroxy-7-azabenzotriazole (HOAt), Isopropyl chloro formate (IPCF), Benzotriazol-1-yl-oxy-tris(dimethyl-amino)-phosphonium hexa fluorophosphate (BOP), benzotriazole-1-yloxytri(pyrrolidino) phosphonium hexa fluoro phosphate (PyBOP), N,N-bis-(2-oxo-3-oxazolidinyl)phosphonic dichloride (BOP-Cl), bromotri(pyrrolidino)phosphonium hexa fluoro phosphate (PyBrOP), 0-(6-Chloro-1-hydrocibenzotriazol-1-yl)-1,1,3,3-tetramethyl uranium tetra fluoroborate (TCTU), chlorotri (pyrrolidino)phosphonium hexafluorophosphate (PyClOP), Ethyl 1,2-dihydro-2-ethoxyquinoline-carboxylate (EEDQ), isobutyl chloro formate (IBCF), 2-succinimido-1,1,3,3-tetramethyluronium tetrafluoro borate (TSTU), 1-Cyano-2-ethoxy-2-oxo ethylidene aminooxy) dimethyl amino morpholino-carbeniumhexafluorophosphate (COMU), 2-(5-norbornen-2,3-dicarboximido)-1,1,3,3-tetramethyluronium tetrafluoroborate (TNTU), propane phosphonic acid anhydride (PPAA), 3-(diethoxy phosphoryloxy)-1,2,3-benzotriazin-4(3H)-one (DEPBT) or its mixture.

17. The process as claimed in claim 2, wherein said solvent used is selected from the group consisting of DMF, DCM, tetrahydrofuran, NMP, DMAC, methanol, ethanol, isopropanol, dichloroethane, 1,4-dioxane, ethyl acetate, acetonitrile, acetone or a mixture thereof.

18. The process as claimed in claim 3, wherein said solvent used is selected from the group consisting of DMF, DCM, tetrahydrofuran, NMP, DMAC, methanol, ethanol, isopropanol, dichloroethane, 1,4-dioxane, ethyl acetate, acetonitrile, acetone or a mixture thereof.

19. The process as claimed in claim 4, wherein said solvent used is selected from the group consisting of DMF, DCM, tetrahydrofuran, NMP, DMAC, methanol, ethanol, isopropanol, dichloroethane, 1,4-dioxane, ethyl acetate, acetonitrile, acetone or a mixture thereof.

20. The process as claimed in claim 5, wherein said solvent used is selected from the group consisting of DMF, DCM, tetrahydrofuran, NMP, DMAC, methanol, ethanol, isopropanol, dichloroethane, 1,4-dioxane, ethyl acetate, acetonitrile, acetone or a mixture thereof.
